(19) United States
(12) Patent Application Publication
(10) Pub. No.: US 2006/0122240 A1
(43) Pub. Date: Jun. 8, 2006

(54) Benzotriazoles and methods of prophylaxis or treatment of metabolic-related disorders thereof

(75) Inventors: Graeme Semple, San Diego, CA (US); Philip J. Skinner, San Diego, CA (US); Martin C. Cherrier, San Diego, CA (US); Peter Webb, San Diego, CA (US); Susan Yoshiko Tamura, San Diego, CA (US)

Correspondence Address: ARENA PHARMACEUTICALS, INC., 6166 NANCY RIDGE DRIVE, SAN DIEGO, CA, 92121, US

(73) Assignee: Arena Pharmaceuticals, Inc., San Diego, CA (US)

(21) Appl. No.: 10/533,799

(22) PCT Filed: Nov. 4, 2003
(86) PCT No.: PCT/US03/35427

§ 371 Date: May 4, 2005

Related U.S. Application Data
(60) Provisional application No. 60/423,819, filed on Nov. 5, 2002.

(51) Int. Cl. C07D 249/16; A61K 31/4192

(52) U.S. Cl. 514/359; 548/260

(57) ABSTRACT

The present invention relates to certain benzotriazole carboxylic acid or ester derivatives of Formula (I), pharmaceutically acceptable salts and solvates thereof, which exhibit useful pharmaceutical properties, for example, as agonists for the GPCR referred to as hRUP38. Also provided by the present invention are pharmaceutical compositions containing compounds of the invention, and methods of using the compounds and compositions of the invention in the prophylaxis or treatment of metabolic-related disorders, including dyslipidemia, atherosclerosis, coronary heart disease, insulin resistance, type 2 diabetes, Syndrome-X and the like. In addition, the present invention also provides for the use of the compounds of the invention in combination with other active agents such as those belonging to the class of -glucosidase inhibitors, aldose reductase inhibitors, biguanides, HMG-CoA reductase inhibitors, squalene synthesis inhibitors, fibrates, LDL catabolism enhancers, angiotensin converting enzyme (ACE) inhibitors, insulin secretion enhancers and the like.

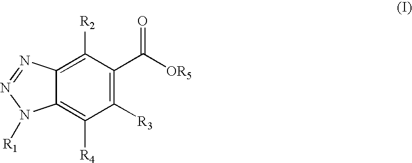

### FIELD OF THE INVENTION

The present invention relates to certain benzotriazole carboxylic acid derivatives, and pharmaceutically acceptable salts thereof, which exhibit useful pharmaceutical properties, for example as agonists for the receptor referred herein as hRUP38. The receptor hRUP38 has been identified to be highly homologous to the receptor hRUP25. The ligand for hRUP25 is nicotinic acid (i.e., niacin). Despite the extremely high homology between these two receptors, a series of receptor specific agonists for the hRUP38 has been identified belonging to the general class of compounds known as benzotriazole carboxylic acids derivatives.

### BACKGROUND OF THE INVENTION

Antilipolytic Agents

Atherosclerosis and stroke are the numbers one and number three leading causes of death of both men and women in the United States. Type 2 diabetes is a public health problem that is serious, widespread and increasing. Elevated levels of low density lipoprotein (LDL) cholesterol or low levels of high density lipoprotein (HDL) cholesterol are, independently, risk factors for atherosclerosis and associated cardiovascular pathologies. In addition, high levels of plasma free fatty acids are associated with insulin resistance and type 2 diabetes. One strategy for decreasing LDL-cholesterol, increasing HDL-cholesterol, and decreasing plasma free fatty acids is to inhibit lipolysis in adipose tissue. This approach involves regulation of hormone sensitive lipase, which is the rate-limiting enzyme in lipolysis. Lipolytic agents increase cellular levels of cAMP, which leads to activation of hormone sensitive lipase within adipocytes. Agents that lower intracellular cAMP levels, by contrast, would be antilipolytic.

It is also worth noting in passing that an increase in cellular levels of cAMP down-regulates the secretion of adiponectin from adipocytes [Delporte, M L et al. Biochem J (2002) July; the disclosure of which is incorporated by reference in its entirety]. Reduced levels of plasma adiponectin have been associated with metabolic-related disorders, including atherosclerosis, coronary heart disease, insulin resistance and type 2 diabetes [Matsuda, M et al. J Biol Chem (2002) July and reviewed therein; the disclosure of which is incorporated by reference in its entirety].

Compounds of the invention inhibit the production and release of free fatty acids from adipose tissue, likely via an inhibition of adenylyl cyclase, a decrease in intracellular cAMP levels, and a concomitant decrease in hormone sensitive lipase activity. Agonists that down-regulate hormone sensitive lipase activity leading to a decrease in plasma free fatty acid levels are likely to have therapeutic value. The consequence of decreasing plasma free fatty acids is two-fold. First, it will ultimately lower LDL-cholesterol and raise HDL-cholesterol levels, independent risk factors, thereby reducing the risk of mortality due to cardiovascular incidence subsequent to atheroma formation. Second, it will provide an increase in insulin sensitivity in individuals with insulin resistance or type 2 diabetes.

Agonists of antilipolytic GPCRs having limited tissue distribution beyond adipose may be especially valuable in view of the diminished opportunity for potentially undesirable side-effects.

This application is related to U.S. Provisional Patent Application Ser. No. 60/423,819 that is incorporated herein by reference in its entirety.

### SUMMARY OF THE INVENTION

One aspect of the present invention encompasses benzotriazole carboxylic acid and ester derivatives as shown in Formula (I):

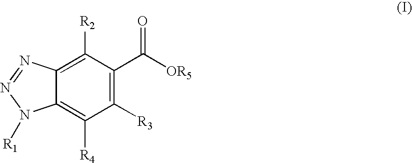

wherein:

R₁ is C₁₋₈ alkyl, C₃₋₆ cycloalkyl or C₁₋₆ haloalkyl, wherein the C₁₋₈ alkyl, C₃₋₆ cycloalkyl and C₁₋₆ haloalkyl groups are optionally substituted with 1, 2, 3 or 4 substituents selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₄ alkylsulfinyl, C₁₋₄ alkylsulfonyl, C₁₋₆ alkylthio, C₁₋₆ alkylureyl, amino, C₁₋₆ alkylamino, aryl, substituted aryl, C₁₋₆ dialkylamino, carbo C₁alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, heteroaryl, heterocyclyl, hydroxyl, nitro or thiol;

R₂, R₃ and R₄ are independently H, C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro or thiol; and

R₅ is H or C₁₋₆ alkyl; or
a pharmaceutically acceptable salt, solvate or hydrate thereof.

In some embodiments, when R₅ is ethyl, and R₂, R₃ and R₄ are H then R₁ is not methyl or triphenylmethyl.

In some embodiments, when R₅ is n-pentyl, and R₂, R₃ and R₄ are H then R₁ is not n-butyl.

In some embodiments, when R₅ is methyl, and R₂, R₃ and R₄ are H then R₁ is not pyrrolidin-1-ylmethyl, 3-tert-butyl-2-hydroxy-5-methyl-benzyl, methyl, or dimethylaminomethyl.

In some embodiments, when R₅ is methyl, R₂ is carbomethoxy (i.e. —CO₂CH₃), and R₃ and R₄ are both H then R₁ is not methyl.

In some embodiments, when R₂, R₃, R₄ and R₅ are all H then R₁ is not 2-amino-2-carboxy-ethyl, pyrrolidin-1-ylmethyl, isopropyl, methyl, benzyl, n-butyl, or carboxymethyl (i.e., —CH₂CO₂H).

In some embodiments, when R₂, R₄, and R₅ are all H and R₃ is methoxy then R₁ is not methyl.

One aspect of the present invention encompasses benzotriazole carboxylic acid and ester derivatives as shown in Formula (I) wherein:

R₁ is C₃₋₆ cycloalkyl or C₁₋₆ haloalkyl, where the C₃₋₆ cycloalkyl or C₁₋₆ haloalkyl group is optionally substituted with C₁₋₆acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro or thiol;

R₂, R₃ and R₄ are independently H, C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro or thiol; and

R₅ is H or C₁₋₆ alkyl; or

a pharmaceutically acceptable salt, solvate or hydrate thereof.

Another aspect of the present invention encompasses compounds of the group consisting of 1-Cyclopentyl-1H-benzotriazole-5-carboxylic acid; 1-(2′-Butyl)-1H-benzotriazole-5-carboxylic acid; 1-(3′-Pentyl)-1H-benzotriazole-5-carboxylic acid; 1-Cyclohexyl-1H-benzotriazole-5-carboxylic acid; 1-Propyl-1H-benzotriazole-5-carboxylic acid; 1-Cyclopropyl-1H-benzotriazole-5-carboxylic acid; 1-(3′-Isopropoxy-propyl)-1H-benzotriazole-5-carboxylic acid; 1-(Tetrahydro-furan-2′-ylmethyl)-H-benzotriazole-5-carboxylic acid; 1-Cyclobutyl-1H-benzotriazole-5-carboxylic acid; 1-(2-Methoxy-ethyl)-1H-benzotriazole-5-carboxylic acid; 1-(3′Methoxybenzyl)-1H-benzotriazole-5-carboxylic acid; 1-(4′Methoxybenzyl)-1H-benzotriazole-5-carboxylic acid; 1-[2′-(4″-Methoxy-phenyl)-ethylamino]-1H-benzotriazole-5-carboxylic acid; 1-[2′-(3″-Methoxy-phenyl)-ethylamino]-1H-benzotriazole-5-carboxylic acid; 1-(3′,5′-Difluorobenzyl)-1H-benzotriazole-5-carboxylic acid; 1-(2-Ethylsulfanyl-ethyl)-1H-benzotriazole-5-carboxylic acid; 1-t-Butyl-1H-benzotriazole-5-carboxylic acid; 1-(3′-Hydroxy-propyl)-1H-benzotriazole-5-carboxylic acid; 1-(1′,3′-Dimethyl-butyl)-1H-benzotriazole-5-carboxylic acid; 1-(3′,3′-Dimethyl-butyl)-1H-benzotriazole-5-carboxylic acid; 1-Heptyl-1H-benzotriazole-5-carboxylic acid; 1-(2′-Methoxy-1′-methyl-ethyl)-1H-benzotriazole-5-carboxylic acid; 1-(2′-Hydroxy-1′-hydroxymethyl-ethyl)-1H-benzotriazole-5-carboxylic acid; 1-Ethyl-1H-benzotriazole-5-carboxylic acid; 1-Pentyl-1H-benzotriazole-5-carboxylic acid; 1-(2′,2′-Dimethyl-propyl)-1H-benzotriazole-5-carboxylic acid; 1-(2′-Ethoxy-ethyl)-1H-benzotriazole-5-carboxylic acid; 1-(1′,2′-Dimethyl-propyl)-1H-benzotriazole-5-carboxylic acid; 1-Benzhydryl-1H-benzotriazole-5-carboxylic acid; 1-Allyl-1H-benzotriazole-5-carboxylic acid; 1-Butyl-1H-benzotriazole-5-carboxylic acid; 1-(Cyclopropylmethyl)-1H-benzotriazole-5-carboxylic acid; 1-(But-2-ynyl)-1H-benzotriazole-5-carboxylic acid; 1-(4′-Methyl-pentyl)-1H-benzotriazole-5-carboxylic acid; and 1-(3′-Methyl-butyl)-1H-benzotriazole-5-carboxylic acid; or a pharmaceutically acceptable salt or a solvate thereof.

Another aspect of the present invention encompasses certain pharmaceutical compositions comprising a compound of Formula (I) or subgenera thereof in combination with a pharmaceutically acceptable carrier.

Another aspect of the present invention encompasses pharmaceutical compositions, as described herein, further comprising one or more agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

Another aspect of the present invention encompasses methods of modulating a RUP38 receptor comprising contacting the receptor with a therapeutically effective amount of a compound as described herein. In some embodiments, the compound is an agonist of the receptor.

Another aspect of the present invention encompasses methods of modulating a RUP38 receptor in an individual comprising contacting the receptor with a therapeutically effective amount of a compound as described herein. In some embodiments, the modulation treats a metabolic-related disorder.

Another aspect of the present invention encompasses methods of modulating RUP38 receptor function in a cell, tissue or individual comprising contacting the cell, tissue or individual with a therapeutically effective amount of a compound as described herein. In some embodiments, the RUP38 receptor function is associated with a metabolic-related disorder.

Another aspect of the present invention encompasses methods of treatment of a metabolic-related disorder comprising administering to an individual in need of such treatment a therapeutically effective amount of a compound or a pharmaceutical composition as described herein.

In some embodiments of the present invention, the metabolic-related disorder is selected from the group consisting of dyslipidemia, atherosclerosis, coronary heart disease, insulin resistance, obesity, impaired glucose tolerance, atheromatous disease, hypertension, stroke, Syndrome X, heart disease and type 2 diabetes. In some embodiments, the metabolic-related disorder is selected from the group consisting of dyslipidemia, atherosclerosis, coronary heart disease, insulin resistance and type 2 diabetes.

In some embodiments of the present invention, the individual is a mammal. In some embodiments, the mammal is a human.

Another aspect of the present invention encompasses methods of producing a pharmaceutical composition comprising admixing a compound as described herein and a pharmaceutically acceptable carrier.

Another aspect of the present invention is a compound according to any of the embodiments described herein or a pharmaceutical composition as described herein for use in a method of treatment of the human or animal body by therapy.

Another aspect of the present invention is a compound according to any of the embodiments described herein or a pharmaceutical composition as described herein for use in a method of treatment of a metabolic-related disorder of the human or animal body by therapy.

Another aspect of the present invention encompasses the use of compounds of Formula (I) for the manufacture of a medicament for use in the treatment of a metabolic-related disorder.

Another aspect of the present invention encompasses the use of compounds of Formula (I) for the manufacture of a medicament for use in the treatment of a metabolic-related disorder selected from the group consisting of dyslipidemia, atherosclerosis, coronary heart disease, insulin resistance, obesity, impaired glucose tolerance, atheromatous disease, hypertension, stroke, Syndrome X, heart disease and type 2 diabetes.

Another aspect of the present invention encompasses the use of compounds of Formula (I) for the manufacture of a medicament for use in the treatment of atherosclerosis.

These and other aspects of the invention disclosed herein will be set forth in greater detail as the patent disclosure proceeds.

### BRIEF DESCRIPTION OF THE DRAWINGS

FIG. 1. FIG. 1 presents screening data via adenylyl cyclase assay for hRUP38. Note that nicotinic acid does not activate inhibition of forskolin stimulated cAMP in hRUP38-expressing CHO cells whereas 1-Isopropyl-1H-benzotriazole-5-carboxylic acid does. 1-Isopropyl-1H-benzotriazole-5-carboxylic acid has no effect on CHO cells expressing hRUP25. The EC₅₀ for 1-Isopropyl-1H-benzotriazole-5-carboxylic acid is 166 nM.

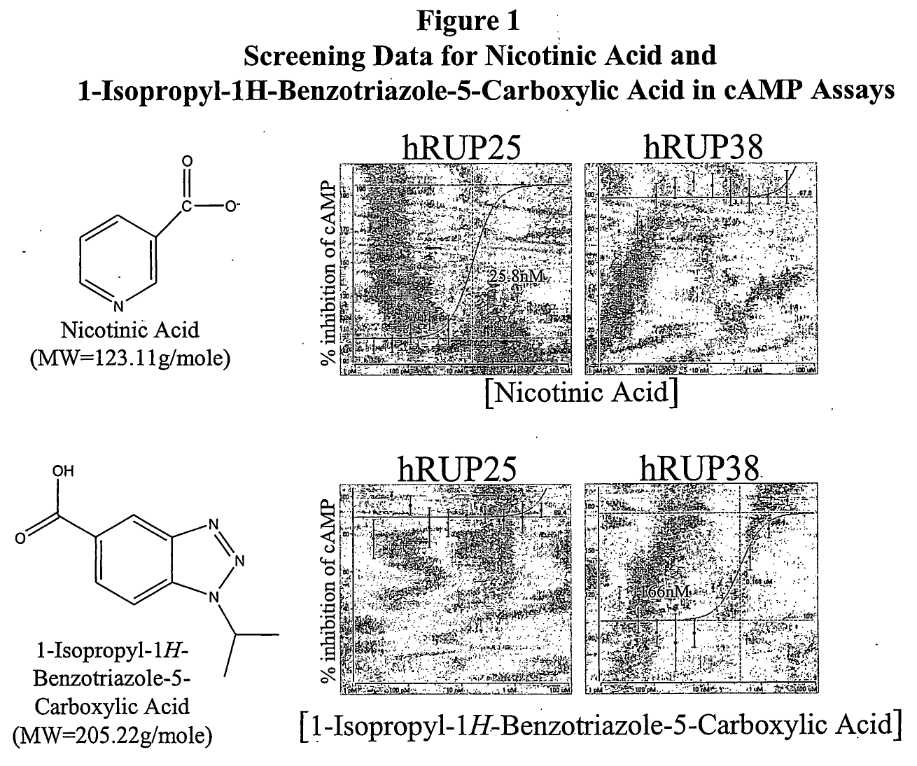

FIG. 2. Nicotinic acid and 1-Isopropyl-1H-benzotriazole-5-carboxylic acid were separately dose-dependently applied to isoproterenol stimulated (100 nM) primary human adipocytes. FIG. 2 illustrates the ability of 1-Isopropyl-1H-benzotriazole-5-carboxylic acid to inhibit isoproterenol stimulated lipolysis in adipocyte primary cultures derived from human subcutaneous fat in a dose-dependant manner comparable to that of nicotinic acid.

### DETAILED DESCRIPTION

One aspect of the present invention encompasses benzotriazole carboxylic acid and ester derivatives as shown in Formula (I):

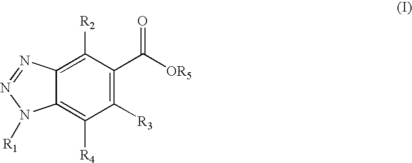

wherein:

R₁ is C₁₋₈ alkyl, C₃₋₆ cycloalkyl or C₁₋₆ haloalkyl, wherein the C₁₋₈ alkyl, C₃₋₆ cycloalkyl and C₁₋₆ haloalkyl groups are optionally substituted with 1, 2, 3 or 4 substituents selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, aryl, substituted aryl, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, heteroaryl, heterocyclyl, hydroxyl, nitro or thiol;

R₂, R₃ and R₄ are independently H, C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro or thiol; and

R₅ is H or C₁₋₆ alkyl; or
a pharmaceutically acceptable salt, solvate or hydrate thereof.

In some embodiments, when R₅ is ethyl, and R₂, R₃ and R₄ are H then R₁ is not methyl or triphenylmethyl.

In some embodiments, when R₅ is n-pentyl, and R₂, R₃ and R₄ are H then R₁ is not n-butyl.

In some embodiments, when R₅ is methyl, and R₂, R₃ and R₄ are H then R₁ is not pyrrolidin-1-ylmethyl, 3-tert-butyl-2-hydroxy-5-methyl-benzyl, methyl, or dimethylaminomethyl. The group pyrrolidin-1-ylmethyl can be represented by the following formula:

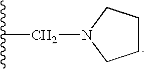

In some embodiments, when R₅ is methyl, R₂ is carbomethoxy (i.e. —CO₂CH₃), and R₃ and R₄ are both H then R₁ is not methyl.

In some embodiments, when R₂, R₃, R₄ and R₅ are all H then R₁ is not 2-amino-2-carboxy-ethyl, pyrrolidin-1-ylmethyl, isopropyl, methyl, benzyl, n-butyl, or carboxymethyl (i.e., —CH₂CO₂H). The group 2-amino-2-carboxy-ethyl can be represented by the following formula:

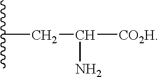

In some embodiments, when R₂, R₄, and R₅ are all H and R₃ is methoxy then R₁ is not methyl.

One aspect of the present invention encompasses benzotriazole carboxylic acid and ester derivatives as shown in Formula (I) wherein:

R₁ is C₃₋₆ cycloalkyl or C₁₋₆ haloalkyl, where the C₃₋₆ cycloalkyl or C₁₋₆ haloalkyl group is optionally substituted with C₁₋₆acyl, C₁₋₆ acyloxy, C₂₋₆ alkenyl, C₁₋₆ alkoxy, C₁₋₆ alkyl, C₁₋₄ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro or thiol;

R₂, R₃ and R₄ are independently H, C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro or thiol; and

R₅ is H or C₁₋₆ alkyl; or

a pharmaceutically acceptable salt, solvate or hydrate thereof.

The present invention also encompasses diastereomers as well as optical isomers, e.g. mixtures of enantiomers including racemic mixtures, as well as individual enantiomers and diastereomers, which arise as a consequence of structural asymmetry in certain compounds of the present invention. In some embodiments, compounds of the present invention are R. In some embodiments, compounds of the present are S. In some embodiments, compounds of the present invention are racemic mixtures.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

In some embodiments, the invention is a compound where R₅ is C₁₋₆alkyl.

In some embodiments, the invention is a compound where R₅ is H and is represented by Formula (Ia) shown below:

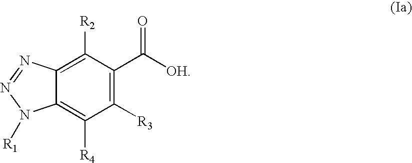

In some embodiments, R₂, R₃ and R₄ are each independently H or halogen. In some embodiments, R₂, R₃ and R₄ are each independently H or F.

In some embodiments, the invention is a compound where R₁ is C₁₋₈ alkyl optionally substituted with substituents selected from the group consisting of C₂₋₆ alkenyl, C₁₋₆ alkoxy, C₂₋₆ alkynyl, C₁₋₆ alkylsulfinyl, C₁₋₆ alkylsulfonyl, C₁₋₆ alkylthio, aryl, substituted aryl, C₃₋₆ cycloalkyl, halogen, C₁₋₆ haloalkoxy, C₁₋₆ haloalkylsulfinyl, C₁₋₆ haloalkylsulfonyl, C₁₋₆ haloalkylthio, heteroaryl, heterocyclyl, and hydroxyl.

In some embodiments, the invention is a compound where R₁ is a C₁₋₈ alkyl group. In some embodiments, R₁ is selected from the group consisting of 2-butyl, 3-pentyl, 1-propyl, t-butyl, 1-butyl, 4-Methyl-pentyl, 3-methyl-butyl, 1,3-dimethyl-butyl, 3,3-dimethyl-butyl, 1-heptyl, ethyl, and 1-pentyl, and 1,2-dimethyl-propyl.

In some embodiments, the invention is a compound where R₁ is a C₁₋₈ alkyl group optionally substituted with a substituted aryl group. In some embodiments, R₁ is selected from the group consisting of 3-methoxy-benzyl, 4-methoxy-benzyl, 4-methoxy-phenyl ethyl, 3-methoxy-phenyl ethyl, 3,5-difluorobenzyl, and benzhydryl.

In some embodiments, the invention is a compound where R₁ is a C₁₋₈ alkyl group optionally substituted with a C₁₋₆ alkoxy group. In some embodiments, R₁ is selected from the group consisting of 3-isopropoxypropyl, 2-methoxy-ethyl, 2-methoxy-1-methyl-ethyl, and 2-ethoxy-ethyl.

In some embodiments, the invention is a compound where R₁ is a C₁₋₈ alkyl group optionally substituted with a heterocyclyl group. In some embodiments, R₁ is tetrahydro-furan-2-ylmethyl.

In some embodiments, the invention is a compound where R₁ is a C₁₋₈ alkyl group optionally substituted with a C₁₋₆ alkylthio group. In some embodiments, R₁ is 2-ethylsulfanyl-ethyl.

In some embodiments, the invention is a compound where R₁ is a C₁₋₈ alkyl group optionally substituted with a hydroxyl group. In some embodiments, R₁ is 3-hydroxy-propyl, 2-hydroxy-1-hydroxymethyl-ethyl, or 2-hydroxy-1-hydroxymethyl-ethyl.

In some embodiments, the invention is a compound where R₁ is a C₁₋₈ alkyl group optionally substituted with a C₂₋₆ alkenyl group. In some embodiments, R₁ is allyl (i.e., —CH₂CH═CH₂).

In some embodiments, the invention is a compound where R₁ is a C₁₋₈ alkyl group optionally substituted with a C₃₋₆ cycloalkyl group. In some embodiments, R₁ is cyclopropylmethyl.

In some embodiments, the invention is a compound where R₁ is a C₁₋₈ alkyl group optionally substituted with a C₂₋₆ alkynyl group. In some embodiments, R₁ is but-2-ynyl.

In some embodiments, the invention is a compound where R₁ is C₃₋₆ cycloalkyl optionally substituted with C₁₋₃ alkoxy, C₁₋₃ alkyl, C₁₋₃ alkylureyl, amino, C₁₋₃ alkylamino, C₁₋₄ dialkylamino, carbo-C₁₋₃-alkoxy, carboxy, cyano, halogen, C₁₋₃ haloalkoxy, C₁₋₃ haloalkyl, hydroxyl, nitro or thiol. Illustrated examples for when R₁ is C₃₋₆ cycloalkyl include cyclopropyl, Formula (Ib); cyclobutyl, Formula (Ic); cyclopentyl, Formula (Id); cyclohexyl, Formula (Ie) and the like.

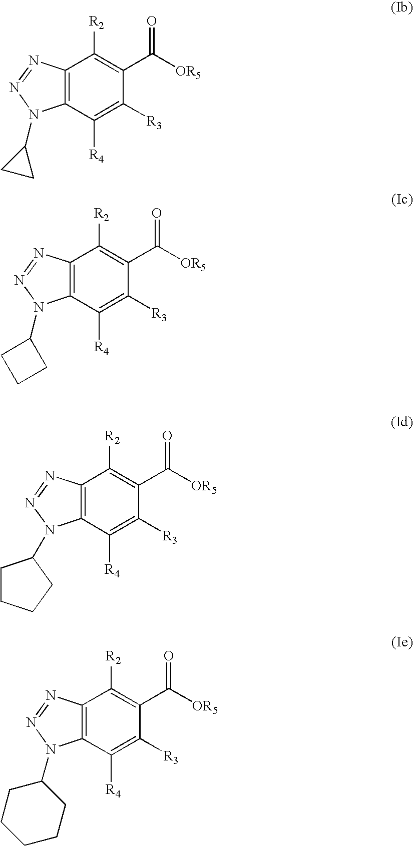

In some embodiments, R₁ is C₃₋₅ cycloalkyl optionally substituted with C₁₋₃ alkyl, halogen, C₁₋₃ haloalkyl or hydroxyl. In some embodiments, R₁ is C₃₋₅ cycloalkyl optionally substituted with C₁₋₃ alkyl or halogen. In some embodiments, R₁ is C₃₋₄ cycloalkyl optionally substituted with 1 to 4 fluorine atoms. In some embodiments, R₁ is a cyclopropyl or cyclobutyl group.

In some embodiments, the invention is a compound where R₁ is C₁₋₆ haloalkyl optionally substituted with C₁₋₃ alkoxy, C₁₋₃ alkylureyl, amino, C₁₋₃ alkylamino, C₁₋₄ dialkylamino, carbo-C₁₋₃-alkoxy, carboxy, cyano, halogen, C₁₋₃ haloalkoxy, hydroxyl, nitro or thiol. In some embodiments, R₁ is C₁₋₅ haloalkyl optionally substituted with amino, C₁₋₃ alkoxy or hydroxyl. In some embodiments, R₁ is CF₃, CF₃CH₂, CF₃CF₂CH₂, (CF₃)₂CH, CF₃CF₂CF₂CH₂ or (CF₃)₂CHCH₂. In some embodiments, R₁ is a 2,2,2-trifluoroethyl, Formula (If); or 2,2,2-trifluoro-1-trifluoromethyl-ethyl group, Formula (Ig).

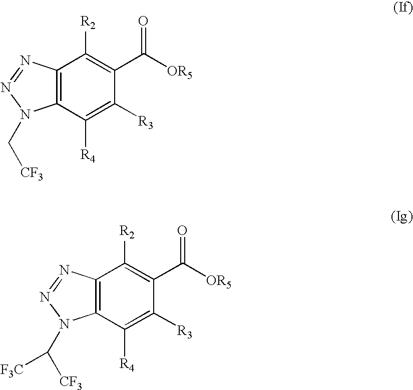

In some embodiments, the invention is a compound where R₂, R₃ and R₄ are independently H, C₁₋₃ alkoxy, C₁₋₃ alkyl, amino, C₁₋₃ alkylamino, C₁₋₄ dialkylamino, halogen, C₁₋₃ haloalkoxy, C₁₋₃ haloalkyl, hydroxyl, nitro or thiol. In some embodiments, R₂, R₃ and R₄ are independently H, C₁₋₃ alkyl, amino, halogen, C₁₋₃ haloalkyl or hydroxyl. In some embodiments, R₂, R₃ and R₄ are independently H, methyl, ethyl, amino, fluorine, chlorine, trifluoromethyl, or hydroxyl.

In some embodiments, the invention is a compound where R₁ is cyclopropyl or cyclobutyl; and R₂, R₃ and R₄ are independently H, methyl, ethyl, amino, fluorine, chlorine, trifluoromethyl, or hydroxyl. In some embodiments, R₂, R₃ and R₄ are independently H, methyl, fluorine, chlorine or trifluoromethyl.

In some embodiments, the invention encompasses a compound wherein R₁ is cyclopropyl and R₅ is H and has the following chemical name. Substitutions are based on the numbering system as shown in Formula (Ih): 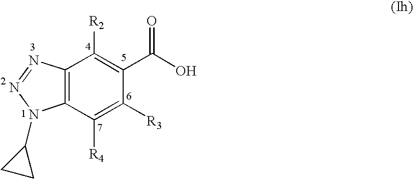
1-Cyclopropyl-1H-benzotriazole-5-carboxylic acid; 1-Cyclopropyl-7-fluoro-1H-benzotriazole-5-carboxylic acid; 1-Cyclopropyl-6-fluoro-1H-benzotriazole-5-carboxylic acid; 1-Cyclopropyl-4-fluoro-1H-benzotriazole-5-carboxylic acid; 1-Cyclopropyl-6,7-difluoro-1H-benzotriazole-5-carboxylic acid; 1-Cyclopropyl-4,7-difluoro-1H-benzotriazole-5-carboxylic acid; 1-Cyclopropyl-4,6-difluoro-1H-benzotriazole-5-carboxylic acid; 1-Cyclopropyl-4,6,7-trifluoro-1H-benzotriazole-5-carboxylic acid; 1-Cyclopropyl-7-chloro-1H-benzotriazole-5-carboxylic acid; 1-Cyclopropyl-6-chloro-1H-benzotriazole-5-carboxylic acid; or 1-Cyclopropyl-4-chloro-1H-benzotriazole-5-carboxylic acid; or a pharmaceutically acceptable salt as described herein below or a solvate as described herein below. Alternatively, a specific compound of the invention as described herein above and below wherein R₅═H the compound may alternatively be an ester where R₅ is C₁₋₆ alkyl. In one embodiment of the present invention, R₅ is C₁₋₄ alkyl, in one embodiment R₅ is C₁₋₂ alkyl, in one embodiment R₅ is C₂₋₆ alkyl, in one embodiment R₅ is C₃₋₆ alkyl, and in one embodiment R₅ is C₄₋₆ alkyl.

In some embodiments, the invention encompasses a compound wherein R₁ is cyclobutyl and R₅ is H and has the following chemical name. Substitutions are based on the numbering system as shown in Formula (Ii):

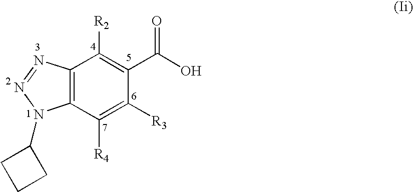

1-Cyclobutyl-1H-benzotriazole-5-carboxylic acid; 1-Cyclobutyl-7-fluoro-1H-benzotriazole-5-carboxylic acid; 1-Cyclobutyl-6-fluoro-1H-benzotriazole-5-carboxylic acid; 1-Cyclobutyl-4-fluoro-1H-benzotriazole-5-carboxylic acid; 1-Cyclobutyl-6,7-difluoro-1H-benzotriazole-5-carboxylic acid; 1-Cyclobutyl-4,7-difluoro-1H-benzotriazole-5-carboxylic acid; 1-Cyclobutyl-4,6-difluoro-1H-benzotriazole-5-carboxylic acid; 1-Cyclobutyl-4,6,7-trifluoro-1H-benzotriazole-5-carboxylic acid; 1-Cyclobutyl-7-chloro-1H-benzotriazole-5-carboxylic acid; 1-Cyclobutyl-6-chloro-1H-benzotriazole-5-carboxylic acid; or 1-Cyclobutyl-4-chloro-1H-benzotriazole-5-carboxylic acid; or a pharmaceutically acceptable salt, solvate or ester thereof.

In some embodiments, the invention encompasses a compound wherein R₁ is 2,2,2-trifluoro-ethyl and R₅ is H and has the following chemical name. Substitutions are based on the numbering system as shown in Formula (Ij):

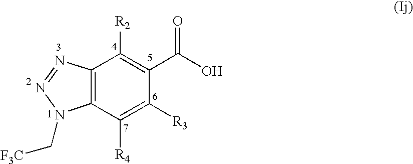

1-(2,2,2-Trifluoro-ethyl)-1H-benzotriazole-5-carboxylic acid; 1-(2,2,2-Trifluoro-ethyl)-7-fluoro-1H-benzotriazole-5-carboxylic acid; 1-(2,2,2-Trifluoro-ethyl)-6-fluoro-1H-benzotriazole-5-carboxylic acid; 1-(2,2,2-Trifluoro-ethyl)-6-fluoro-1H-benzotriazole-5-carboxylic acid; 1-(2,2,2-Trifluoro-ethyl)-6,7-difluoro-1H-benzotriazole-5-carboxylic acid; 1-(2,2,2-Trifluoro-ethyl)-4,7-difluoro-1H-benzotriazole-5-carboxylic acid; 1-(2,2,2-Trifluoro-ethyl)-4,6-difluoro-1H-benzotriazole-5-carboxylic acid; 1-(2,2,2-Trifluoro-ethyl)-4,6,7-trifluoro-1H-benzotriazole-5-carboxylic acid; 1-(2,2,2-Trifluoro-ethyl)-7-chloro-1H-benzotriazole-5-carboxylic acid; 1-(2,2,2-Trifluoro-ethyl)-6-chloro-1H-benzotriazole-5-carboxylic acid; or 1-(2,2,2-Trifluoro-ethyl)-4-chloro-1H-benzotriazole-5-carboxylic acid; or a pharmaceutically acceptable salt, solvate or ester thereof.

In some embodiments, the invention encompasses a compound wherein R₁ is 2,2,2-trifluoro-ethyl and R₅ is H and has the following chemical name. Substitutions are based on the numbering system as shown in Formula (Ik): 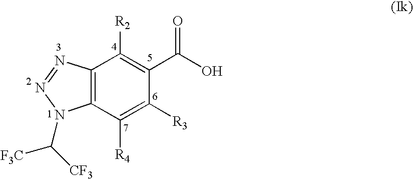
1-(2,2,2-Trifluoro-1-trifluoromethyl-ethyl)-1H-benzotriazole-5-carboxylic acid; 1-(2,2,2-Trifluoro-1-trifluoromethyl-ethyl)-7-fluoro-1H-benzotriazole-5-carboxylic acid; 1-(2,2,2-Trifluoro-1-trifluoromethyl-ethyl)-6-fluoro-1H-benzotriazole-5-carboxylic acid; 1-(2,2,2-Trifluoro-1-trifluoromethyl-ethyl)₄-fluoro-1H-benzotriazole-5-carboxylic acid; 1-(2,2,2-Trifluoro-1-trifluoromethyl-ethyl)-6,7-difluoro-1H-benzotriazole-5-carboxylic acid; 1-(2,2,2-Trifluoro-1-trifluoromethyl-ethyl)-4,7-difluoro-1H-benzotriazole-5-carboxylic acid; 1-(2,2,2-Trifluoro-1-trifluoromethyl-ethyl)-4,6-difluoro-1H-benzotriazole-5-carboxylic acid; 1-(2,2,2-Trifluoro-1-trifluoromethyl-ethyl)-4,6,7-trifluoro-1H-benzotriazole-5-carboxylic acid; 1-(2,2,2-Trifluoro-1-trifluoromethyl-ethyl)-7-chloro-1H-benzotriazole-5-carboxylic acid; 1-(2,2,2-Trifluoro-1-trifluoromethyl-ethyl)-6-chloro-1H-benzotriazole-5-carboxylic acid; or 1-(2,2,2-Trifluoro-1-trifluoromethyl-ethyl)₄-chloro-1H-benzotriazole-5-carboxylic acid; or a pharmaceutically acceptable salt, solvate or ester thereof.

One aspect of the present invention encompasses a pharmaceutical composition according to any one of the compound embodiments of Formula (I) in combination with a pharmaceutically acceptable carrier.

One aspect of the present invention encompasses a pharmaceutical composition comprising a compound of Formula (I):

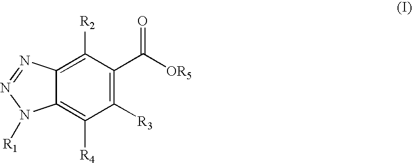

wherein:
R₁ is H, C₁₋₆ alkyl, C₃₋₆ cycloalkyl or C₁₋₆ haloalkyl, wherein each C₁₋₆ alkyl, C₃₋₆ cycloalkyl or C₁₋₆ haloalkyl group is optionally substituted with C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro or thiol;
R₂, R₃ and R₄ are independently H, C₁₋₆ acyl, C₁₋₆ acyloxy, C₂₋₆alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro or thiol; and
R₅ is H or C₁₋₆ alkyl; or
a pharmaceutically acceptable salt, solvate or hydrate thereof in combination with a pharmaceutically acceptable carrier.

In some embodiments, the pharmaceutical composition is where R₁ is C₁₋₆ alkyl optionally substituted with C₁₋₃ alkoxy, C₁₋₃ alkylureyl, amino, C₁₋₃ alkylamino, C₁₋₄ dialkylamino, carbo-C₁₋₃-alkoxy, carboxy, cyano, C₃₋₅ cycloalkyl, halogen, C₁₋₃ haloalkoxy, hydroxyl, nitro or thiol. In some embodiments, R₁ is C₁₋₆ alkyl optionally substituted with C₁₋₃ alkoxy, amino, C₃₋₅ cycloalkyl or hydroxyl. In some embodiments, R₁ is C₁₋₆ alkyl further substituted with C₃₋₅ cycloalkyl. In some embodiments, R₁ is cyclopropylmethyl as shown by Formula (Im), dicyclopropylmethyl as shown by Formula (In), 1-(1-cyclopropyl-ethyl) as shown by Formula (Io), 1-(2-cyclopropyl-ethyl) as shown in Formula (Ip), cyclobutylmethyl as shown by Formula (Iq), 1-(1-cyclobutyl-ethyl) as shown by Formula (Ir) or 1-(2-cyclobutyl-ethyl) as shown by Formula (Is).

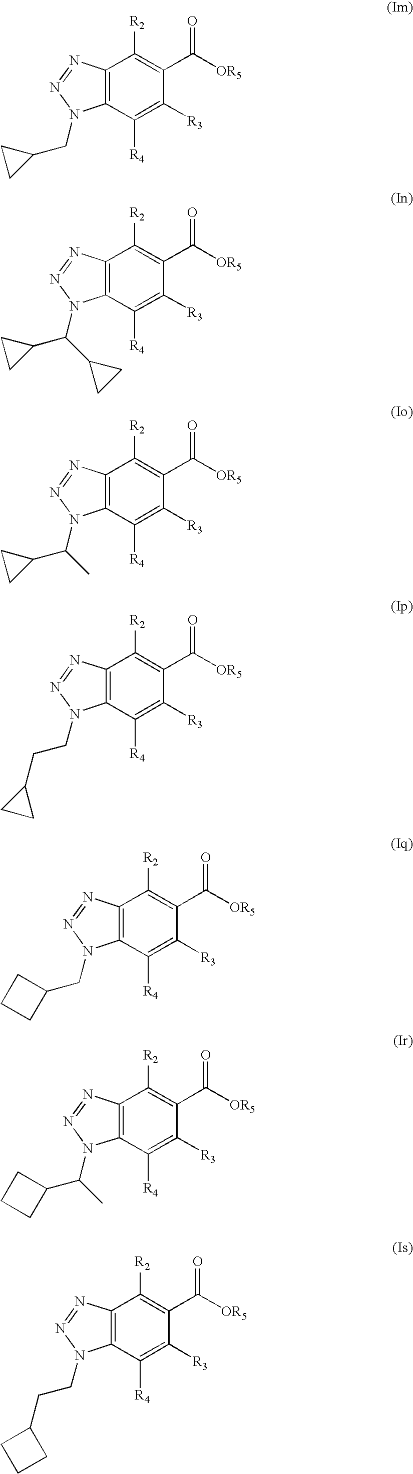

In some embodiments, the pharmaceutical composition is where R₁ is C₁₋₆ alkyl. In some embodiments, R₁ is CH₃, CH₃CH₂, CH₃CH₂CH₂, (CH₃)₂CH, CH₃CH₂CH₂CH₂, (CH₃)₂CHCH₂, CH₃CH₂CH(CH₃), (CH₃)₃C, CH₃CH₂CH₂CH₂CH₂, (CH₃)₂CHCH₂CH₂, CH₃CH₂CH(CH₃)CH₂, CH₃CH₂CH₂CH(CH₃), (CH₃)₃CCH₂, CH₃CH₂C(CH₃)₂ or CH₃CHCH₃CHCH₃. In some embodiments, R₁ is CH₃, CH₃CH₂, CH₃CH₂CH₂, (CH₃)₂CH, CH₃CH₂CH₂CH₂, (CH₃)₂CHCH₂, CH₃CH₂CH(CH₃), or (CH₃)₃C.

In some embodiments, the pharmaceutical composition is where R₁ is C₁₋₆ haloalkyl optionally substituted with C₁₋₃ alkoxy, C₁₋₃ alkylureyl, amino, C₁₋₃ alkylamino, C₁₋₄ dialkylamino, carbo-C₁₋₃-alkoxy, carboxy, cyano, C₁₋₃ haloalkoxy, hydroxyl, nitro or thiol. In some embodiments, R₁ is C₁₋₅ is haloalkyl optionally substituted with amino, C₁₋₃ alkoxy or hydroxyl. In some embodiments, R₁ is CF₃, CF₃CH₂, CF₃CF₂CH₂, (CF₃)₂CH, CF₃CF₂CF₂CH₂ or (CF₃)₂CHCH₂.

In some embodiments, the pharmaceutical composition is where R₂, R₃ and R₄ are independently H, C₁₋₄ alkoxy, C₁₋₄ alkyl, C₁₋₄ alkylthio, amino, cyano, C₃₋₅ cycloalkyl, halogen, C₁₋₃ haloalkoxy, C₁₋₃ haloalkyl, hydroxyl, nitro or thiol. In some embodiments, R₂, R₃ and R₄ are independently H, C₁₋₂ alkoxy, C₁₋₂ alkyl, C₁₋₂ alkylthio, amino, cyano, C₃₋₅ cycloalkyl, halogen, C₁₋₂ haloalkoxy, C₁₋₂ haloalkyl, hydroxyl, nitro or thiol. In some embodiments, R₂, R₃ and R₄ are independently H, methoxy, methyl, methylsulfide, amino, cyano, cyclopropyl, cyclobutyl, fluorine atom, chlorine atom, bromine atom, trifluoromethoxy, difluoromethoxy, fluoromethoxy, trifluoromethyl, difluoromethyl, hydroxyl, or thiol. In some embodiments, R₂, R₃ and R₄ are independently H, methoxy, methyl, methylsulfide, amino, cyano, fluorine atom, chlorine atom, trifluoromethoxy, difluoromethoxy, trifluoromethyl, difluoromethyl, or hydroxyl.

In some embodiments, the pharmaceutical composition is where R₁ is C₃₋₆ cycloalkyl optionally substituted with C₁₋₃ alkoxy, C₁₋₃ alkyl, C₁₋₃ alkylureyl, amino, C₁₋₃ alkylamino, C₁₋₄ dialkylamino, carbo-C₁₋₃-alkoxy, carboxy, cyano, halogen, C₁₋₃ haloalkoxy, C₁₋₃ haloalkyl, hydroxyl, nitro or thiol. In some embodiments, R₁ is C₃₋₅ cycloalkyl optionally substituted with C₁₋₃ alkyl, halogen, C₁₋₃ haloalkyl or hydroxyl. In some embodiments, R₁ is C₃₋₅ cycloalkyl optionally substituted with C₁₋₃ alkyl or halogen. In some embodiments, R₁ is C₃₋₄ cycloalkyl optionally substituted with 1 to 7 fluorine atoms. In some embodiments, R₁ is a cyclopropyl or cyclobutyl group.

In some embodiments, the pharmaceutical composition is where R₁ is C₁₋₆ alkyl; and R₂, R₃ and R₄ are independently H, C₁₋₃ alkoxy, C₁₋₃ alkyl, C₁₋₃ alkylureyl, amino, C₁₋₃ alkylamino, C₁₋₄ dialkylamino, carbo-C₁₋₃-alkoxy, carboxy, cyano, halogen, C₁₋₃ haloalkoxy, C₁₋₃ haloalkyl, hydroxyl, nitro or thiol. In some embodiments, R₁ is C₁₋₄ alkyl; and R₂, R₃ and R₄ are independently H, C₁₋₃ alkyl, amino, halogen, C₁₋₃ haloalkyl or hydroxyl. In some embodiments, R₂, R₃ and R₄ are independently H, methyl, ethyl, amino, fluorine, chlorine, trifluoromethyl, or hydroxyl. In some embodiments, R₂, R₃ and R₄ are independently H, methyl, amino, fluorine, trifluoromethyl or hydroxyl.

In some embodiments, the pharmaceutical composition is where R₁ is C₃₋₆ cycloalkyl; and R₂, R₃ and R₄ are independently H, C₁₋₃ alkoxy, C₁₋₃ alkyl, C₁₋₃ alkylureyl, amino, C₁₋₃ alkylamino, C₁₋₄ dialkylamino, carbo-C₁₋₃-alkoxy, carboxy, cyano, halogen, C₁₋₃ haloalkoxy, C₁₋₃ haloalkyl, hydroxyl, nitro or thiol. In some embodiments, R₁ is C₃₋₄ cycloalkyl; and R₂, R₃ and R₄ are independently H, C₁₋₃ alkyl, amino, halogen, C₁₋₃ haloalkyl or hydroxyl. In some embodiments, R₂, R₃ and R₄ are independently H, methyl, ethyl, amino, fluorine, chlorine, trifluoromethyl, or hydroxyl. In some embodiments, R₂, R₃ and R₄ are independently H, methyl, amino, fluorine, trifluoromethyl or hydroxyl.

In some embodiments, the pharmaceutical composition is where R₁ is C₁₋₆ haloalkyl; and R₂, R₃ and R₄ are independently H, C₁₋₃ alkoxy, C₁₋₃ alkyl, C₁₋₃ alkylureyl, amino, C₁₋₃ alkylamino, C₁₋₄ dialkylamino, carbo-C₁₋₃-alkoxy, carboxy, cyano, halogen, C₁₋₃ haloalkoxy, C₁₋₃ haloalkyl, hydroxyl, nitro or thiol. In some embodiments, R₁ is C₁₋₃ haloalkyl; and R₂, R₃ and R₄ are independently H, C₁₋₃ alkyl, amino, halogen, C₁₋₃ haloalkyl or hydroxyl. In some embodiments, R₂, R₃ and R₄ are independently H, methyl, ethyl, amino, fluorine, chlorine, trifluoromethyl, or hydroxyl. In some embodiments, R₂, R₃ and R₄ are independently H, methyl, amino, fluorine, trifluoromethyl or hydroxyl.

In one aspect of the present invention, the pharmaceutical composition further comprising one or more agents selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

In some embodiments of the invention the pharmaceutical composition further comprises a α-glucosidase inhibitor. In some embodiments, the α-glucosidase inhibitor is acarbose, voglibose or miglitol. In some embodiments, the α-glucosidase inhibitor is voglibose.

In some embodiments of the invention the pharmaceutical composition further comprises an aldose reductase inhibitor. In some embodiments, the aldose reductase inhibitor is tolurestat; epalrestat; imirestat; zenarestat; zopolrestat; or sorbinil.

In some embodiments of the invention the pharmaceutical composition further comprises a biguanide. In some embodiments, the biguanide is phenformin, metformin or buformin. In some embodiments, the biguanide is metformin.

In some embodiments of the invention the pharmaceutical composition further comprises a HMG-CoA reductase inhibitor. In some embodiments, the HMG-CoA reductase inhibitor is rosuvastatin, pravastatin, simvastatin, lovastatin, atorvastatin, fluvastatin or cerivastatin.

In some embodiments of the invention the pharmaceutical composition further comprises a fibrate. In some embodiments, the fibrate is bezafibrate, beclobrate, binifibrate, ciplofibrate, clinofibrate, clofibrate, clofibric acid, etofibrate, fenofibrate, gemfibrozil, nicofibrate, pirifibrate, ronifibrate, simfibrate, or theofibrate.

In some embodiments of the invention the pharmaceutical composition further comprises an angiotensin converting enzyme inhibitor. In some embodiments, the angiotensin converting enzyme inhibitor is captopril, enalapril, alacepril, delapril; ramipril, lisinopril, imidapril, benazepril, ceronapril, cilazapril, enalaprilat, fosinopril, moveltopril, perindopril, quinapril, spirapril, temocapril or trandolapril.

In some embodiments of the invention the pharmaceutical composition further comprises an insulin secretion enhancer. In some embodiments, the insulin secretion enhancer is tolbutamide; chlorpropamide; tolazamirde; acetohexamide; glycopyramide; glibenclamide; gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolcyclamide, glimepiride, nateglinide, or mitiglinide.

In some embodiments of the invention the pharmaceutical composition further comprises a thiazolidinedione. In some embodiments, the thiazolidinedione is rosiglitazone or pioglitazone. In some embodiments, the thiazolidinedione is rosiglitazone.

One aspect of the present invention encompasses a method of prophylaxis of a metabolic disorder comprising administering to a patient in need of such administration a prophylactically effective amount of a compound or a pharmaceutical composition according to any of the embodiments disclosed herein. In some embodiments, the metabolic disorder is dyslipidemia, atherosclerosis, coronary heart disease, insulin resistance, obesity, impaired glucose tolerance, atheromatous disease, hypertension, stroke, Syndrome X, heart disease and type 2 diabetes. In some embodiments, the metabolic disorder is dyslipidemia, atherosclerosis, coronary heart disease, insulin resistance and type 2 diabetes.

In some embodiments, compounds of the invention have at least about 2 times greater selectivity for hRUP38 compared to hRUP25 (i.e., EC₅₀ hRUP25÷EC₅₀ hRUP38=about 2). In some embodiments, compounds of the invention have at least about 4 times greater selectivity for hRUP38 compared to hRUP25. In some embodiments, compounds of the invention have about 6 times greater selectivity for hRUP38 compared to hRUP25.

One aspect of the present invention encompasses a method of treatment of a metabolic disorder comprising administrating to a patient in need of such administration a therapeutically effective amount of a compound or a pharmaceutical composition according to any of the embodiments disclosed herein. In some embodiments, the metabolic disorder is dyslipidemia, atherosclerosis, coronary heart disease, insulin resistance, obesity, impaired glucose tolerance, atheromatous disease, hypertension, stroke, Syndrome X, heart disease and type 2 diabetes. In some embodiments, the metabolic disorder is dyslipidemia, atherosclerosis, coronary heart disease, insulin resistance and type 2 diabetes.

One aspect of the present invention encompasses the use of a compound for production of a medicament for use in prophylaxis or treatment of a metabolic disorder wherein the compound is of Formula (I):

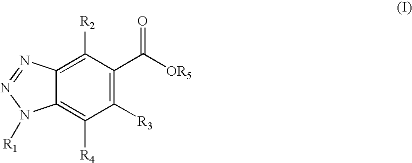

wherein:

R₁ is H, C₁₋₆ alkyl, C₃₋₆ cycloalkyl or C₁₋₆ haloalkyl, where the C₁₋₆ alkyl, C₃₋₆ cycloalkyl or C₁₋₆ haloalkyl group is optionally substituted with C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro or thiol;

R₂, R₃ and R₄ are independently H, C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro or thiol; and R₅ is H or C₁₋₆ alkyl; or

a pharmaceutically acceptable salt, solvate or hydrate thereof

Some embodiments of the present invention encompass the use of a compound of the invention for the production of a medicament wherein R₁ is C₁₋₆ alkyl optionally substituted with C₁₋₃ alkoxy, C₁₋₃ alkylureyl, amino, C₁₋₃ alkylamino, C₁₋₄ dialkylamino, carbo-C₁₋₃-alkoxy, carboxy, cyano, C₃₋₅ cycloalkyl, halogen, C₁₋₃ haloalkoxy, hydroxyl, nitro or thiol. In some embodiments, R₁ is C₁₋₆ alkyl optionally substituted with C₁₋₃ alkoxy, amino, C₃₋₅ cycloalkyl or hydroxyl. In some embodiments, R₁ is C₁₋₆ alkyl further substituted with C₃₋₅ cycloalkyl. In some embodiments, R₁ is cyclopropylmethyl [Formula (Im)], dicyclopropylmethyl [Formula (In)], 1-(1-cyclopropyl-ethyl) [Formula (Io)], 1-(2-cyclopropyl-ethyl) [Formula (Ip)], cyclobutylmethyl [Formula (Iq)], 1-(1-cyclobutyl-ethyl) [Formula (Ir)] or 1-(2-cyclobutyl-ethyl) [Formula (Is].

Some embodiments of the present invention encompass the use of a compound disclosed herein where R₁ is C₁₋₆ alkyl. In some embodiments, R₁ is CH₃, CH₃CH₂, CH₃CH₂CH₂, (CH₃)₂CH, CH₃CH₂CH₂CH₂, (CH₃)₂CHCH₂, CH₃CH₂CH(CH₃), (CH₃)₃C, CH₃CH₂CH₂CH₂CH₂, (CH₃)₂CHCH₂CH₂, CH₃CH₂CH(CH₃)CH₂, CH₃CH₂CH₂CH(CH₃), (CH₃)₃CCH₂, CH₃CH₂C(CH₃)₂ or CH₃CHCH₃CHCH₃. In some embodiments, R₁ is CH₃, CH₃CH₂, CH₃CH₂CH₂, (CH₃)₂CH, CH₃CH₂CH₂CH₂, (CH₃)₂CHCH₂, CH₃CH₂CH(CH₃), or (CH₃)₃C.

Some embodiments of the present invention encompass the use of a compound disclosed herein where R₁ is C₁₋₆ haloalkyl optionally substituted with C₁₋₃ alkoxy, C₁₋₃ alkylureyl, amino, C₁₋₃ alkylamino, C₁₋₄ dialkylamino, carbo-C₁₋₃-alkoxy, carboxy, cyano, C₁₋₃ haloalkoxy, hydroxyl, nitro or thiol. In some embodiments, R₁ is C₁₋₅ haloalkyl optionally substituted with amino, C₁₋₃ alkoxy or hydroxyl. In some embodiments, R₁ is CF₃, CF₃CH₂ CF₃CF₂CH₂, (CF₃)₂CH, CF₃CF₂CF₂CH₂ or (CF₃)₂CHCH₂.

Some embodiments of the present invention encompass the use of a compound disclosed herein where R₂, R₃ and R₄ are independently H; C₁₋₄ alkoxy, C₁₋₄ alkyl, C₁₋₄ alkylthio, amino, cyano, C₃₋₅ cycloalkyl, halogen, C₁₋₃ haloalkoxy, C₁₋₃ haloalkyl, hydroxyl, nitro or thiol. In some embodiments, R₂, R₃ and R₄ are independently H, C₁₋₂ alkoxy, C₁₋₂ alkyl, C₁₋₂ alkylthio, amino, cyano, C₃₋₅ cycloalkyl, halogen, C₁₋₂ haloalkoxy, C₁₋₂ haloalkyl, hydroxyl, nitro or thiol. In some embodiments, R₂, R₃ and R₄ are independently H, methoxy, methyl, methylsulfide, amino, cyano, cyclopropyl, cyclobutyl, fluorine atom, chlorine atom, bromine atom, trifluoromethoxy, difluoromethoxy, fluoromethoxy, trifluoromethyl, difluoromethyl, hydroxyl, or thiol. In some embodiments, R₂, R₃ and R₄ are independently H, methoxy, methyl, methylsulfide, amino, cyano, fluorine atom, chlorine atom, trifluoromethoxy, difluoromethoxy, trifluoromethyl, difluoromethyl, or hydroxyl.

Some embodiments of the present invention encompass the use of a compound disclosed herein where R₁ is C₃₋₆ cycloalkyl optionally substituted with C₁₋₃ alkoxy, C₁₋₃ alkyl, C₁₋₃ alkylureyl, amino, C₁₋₃ alkylamino, C₁₋₄ dialkylamino, carbo-C₁₋₃-alkoxy, carboxy, cyano, halogen, C₁₋₃ haloalkoxy, C₁₋₃ haloalkyl, hydroxyl, nitro or thiol. In some embodiments, R₁ is C₃₋₅ cycloalkyl optionally substituted with C₁₋₃ alkyl, halogen, C₁₋₃ haloalkyl or hydroxyl. In some embodiments, R₁ is C₃₋₅ cycloalkyl optionally substituted with C₁₋₃ alkyl or halogen. In some embodiments, R₁ is C₃₋₄ cycloalkyl optionally substituted with 1 to 7 fluorine atoms. In some embodiments, R₁ is a cyclopropyl or cyclobutyl group.

Some embodiments of the present invention encompass the use of a compound disclosed herein where R₁ is C₁₋₆ alkyl; and R₂, R₃ and R₄ are independently H, C₁₋₃ alkoxy, C₁₋₃ alkyl, C₁₋₃ alkylureyl, amino, C₁₋₃ alkylamino, C₁₋₃ dialkylamino, carbo-C₁₋₃-alkoxy, carboxy, cyano, halogen, C₁₋₃ haloalkoxy, C₁₋₃ haloalkyl, hydroxyl, nitro or thiol. In some embodiments, R₁ is C₁₋₄ alkyl; and R₂, R₃ and R₄ are independently H, C₁₋₃ alkyl, amino, halogen, C₁₋₃ haloalkyl or hydroxyl. In some embodiments, R₂, R₃ and R₄ are independently H, methyl, ethyl, amino, fluorine, chlorine, trifluoromethyl, or hydroxyl. In some embodiments, R₂, R₃ and R₄ are independently H, methyl, amino, fluorine, trifluoromethyl or hydroxyl.

Some embodiments of the present invention encompass the use of a compound disclosed herein where R₁ is C₃₋₆ cycloalkyl; and R₂, R₃ and R₄ are independently H, C₁₋₃ alkoxy, C₁₋₃ alkyl, C₁₋₃ alkylureyl, amino, C₁₋₃ alkylamino, C₁₋₄ dialkylamino, carbo-C₁₋₃-alkoxy, carboxy, cyano, halogen, C₁₋₃ haloalkoxy, C₁₋₃ haloalkyl, hydroxyl, nitro or thiol. In some embodiments, R₁ is C₃₋₄ cycloalkyl; and R₂, R₃ and R₄ are independently H, C₁₋₃ alkyl, amino, halogen, C₁₋₃ haloalkyl or hydroxyl. In some embodiments, R₂, R₃ and R₄ are independently H, methyl, ethyl, amino, fluorine, chlorine, trifluoromethyl, or hydroxyl. In some embodiments, R₂, R₃ and R₄ are independently H, methyl, amino, fluorine, trifluoromethyl or hydroxyl.

Some embodiments of the present invention encompass the use of a compound disclosed herein where R₁ is C₁₋₆ haloalkyl; and R₂, R₃ and R₄ are independently H, C₁₋₃ alkoxy, C₁₋₃ alkyl, C₁₋₃ alkylureyl, amino, C₁₋₃ alkylamino, C₁₋₄ dialkylamino, carbo-C₁₋₃-alkoxy, carboxy, cyano, halogen, C₁₋₃ haloalkoxy, C₁₋₃ haloalkyl, hydroxyl, nitro or thiol. In some embodiments, R₁ is C₁₋₃ haloalkyl; and R₂, R₃ and R₄ are independently H, C₁₋₃ alkyl, amino, halogen, C₁₋₃ haloalkyl or hydroxyl. In some embodiments, R₂, R₃ and R₄ are independently H, methyl, ethyl, amino, fluorine, chlorine, trifluoromethyl, or hydroxyl. In some embodiments, R₂, R₃ and R₄ are independently H, methyl, amino, fluorine, trifluoromethyl or hydroxyl.

One aspect of the invention encompasses the use according to embodiments disclosed herein further comprising one or more agents selected from the group consisting of a α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

Some embodiments of the present invention encompass the use of a compound of the invention for the production of a medicament further comprising a α-glucosidase inhibitor. In some embodiments, the α-glucosidase inhibitor is acarbose, voglibose or miglitol. In some embodiments, the α-glucosidase inhibitor is voglibose.

Some embodiments of the present invention encompass the use of a compound of the invention for the production of a medicament further comprising an aldose reductase inhibitor. In some embodiments, the aldose reductase inhibitor is tolurestat; epalrestat; imirestat; zenarestat; zopolrestat; or sorbinil.

Some embodiments of the present invention encompass the use of a compound of the invention for the production of a medicament further comprising a biguanide. In some embodiments, the biguanide is phenformin, metformin or buformin. In some embodiments, the biguanide is metformin.

Some embodiments of the present invention encompass the use of a compound of the invention for the production of a medicament further comprising a HMG-CoA reductase inhibitor. In some embodiments, the HMG-CoA reductase inhibitor is rosuvastatin, pravastatin, simvastatin, lovastatin, atorvastatin, fluvastatin or cerivastatin.

Some embodiments of the present invention encompass the use of a compound of the invention for the production of a medicament further comprising a fibrate. In some embodiments, the fibrate is bezafibrate, beclobrate, binifibrate, ciplofibrate, clinofibrate, clofibrate, clofibric acid, etofibrate, fenofibrate, gemfibrozil, nicofibrate, pirifibrate, ronifibrate, simfibrate, or theofibrate.

Some embodiments of the present invention encompass the use of a compound of the invention for the production of a medicament further comprising an angiotensin converting enzyme inhibitor. In some embodiments, the angiotensin converting enzyme inhibitor is captopril, enalapril, alacepril, delapril; ramipril, lisinopril, imidapril, benazepril, ceronapril, cilazapril, enalaprilat, fosinopril, moveltopril, perindopril, quinapril, spirapril, temocapril or trandolapril.

Some embodiments of the present invention encompass the use of a compound of the invention for the production of a medicament further comprising an insulin secretion enhancer. In some embodiments, the insulin secretion enhancer is tolbutamide; chlorpropamide; tolazamide; acetohexamide; glycopyramide; glibenclamide; gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolcyclamide, glimepiride, nateglinide, or mitiglinide.

Some embodiments of the present invention encompass the use of a compound of the invention for the production of a medicament further comprising a thiazolidinedione. In some embodiments, the thiazolidinedione is rosiglitazone or pioglitazone. In some embodiments, the thiazolidinedione is rosiglitazone.

Some embodiments of the present invention encompass the use of a compound of the invention for the production of a medicament wherein the metabolic disorder is dyslipidemia, atherosclerosis, coronary heart disease, insulin resistance, obesity, impaired glucose tolerance, atheromatous disease, hypertension, stroke, Syndrome X, heart disease and type 2 diabetes. In some embodiments, the metabolic disorder is dyslipidemia, atherosclerosis, coronary heart disease, insulin resistance and type 2 diabetes.

One aspect of the present invention encompasses a process for preparing a composition comprising admixing a compound and a pharmaceutically acceptable carrier wherein the compound is of Formula (I):

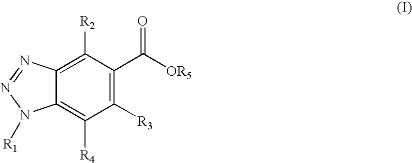

wherein:

R₁ is H, C₁₋₆ alkyl, C₃₋₆ cycloalkyl or C₁₋₆ haloalkyl, where the C₁₋₆ alkyl, C₃₋₆ cycloalkyl or C₁₋₆ haloalkyl group is optionally substituted with C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro or thiol;

R₂, R₃ and R₄ are independently H, C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro or thiol; and

R₅ is H or C₁₋₆ alkyl; or

a pharmaceutically acceptable salt, solvate or hydrate thereof.

These and other aspects of the invention disclosed herein will be set forth in greater detail as the patent disclosure proceeds.

Definitions

The scientific literature has adopted a number of terms, for consistency and clarity, the following definitions will be used throughout this patent document.

AGONISTS shall mean materials (e.g., ligands, candidate compounds) that activate an intracellular response when they bind to the receptor. In some embodiments, AGONISTS are those materials not previously known to activate the intracellular response when they bind to the receptor (e.g. to enhance GTPγS binding to membranes or to lower intracellular cAMP level). In some embodiments, AGONISTS are those materials not previously known to inhibit lipolysis when they bind to the receptor.

AMINO ACID ABBREVIATIONS used herein are set out in TABLE 1:

TABLE 1

| | | |
|---|---|---|
| ALANINE | ALA | A |
| ARGININE | ARG | R |
| ASPARAGINE | ASN | N |
| ASPARTIC ACID | ASP | D |
| CYSTEINE | CYS | C |
| GLUTAMIC ACID | GLU | E |
| GLUTAMINE | GLN | Q |
| GLYCINE | GLY | G |
| HISTIDINE | HIS | H |
| ISOLEUCINE | ILE | I |
| LEUCINE | LEU | L |
| LYSINE | LYS | K |
| METHIONINE | MET | M |
| PHENYLALANINE | PHE | F |
| PROLINE | PRO | P |
| SERINE | SER | S |
| THREONINE | THR | T |
| TRYPTOPHAN | TRP | W |
| TYROSINE | TYR | Y |
| VALINE | VAL | V |

ANTAGONISTS shall mean materials (e.g., ligands, candidate compounds) that competitively bind to the receptor at the same site as the agonists but which do not activate an intracellular response, and can thereby inhibit the intracellular responses elicited by agonists. ANTAGONISTS do not diminish the baseline intracellular response in the absence of an agonist. In some embodiments, ANTAGONISTS are those materials not previously known to compete with an agonist to inhibit the cellular response when they bind to the receptor, e.g. wherein the cellular response is GTPγS binding to membranes or to the lowering of intracellular cAMP level.

ATHEROSCLEROSIS is intended herein to encompass disorders of large and medium-sized arteries that result in the progressive accumulation within the intima of smooth muscle cells and lipids.

CHEMICAL GROUP, MOIETY or RESIDUE shall have the following meaning in the specification and Formulae described herein:
The term “C₁₋₆ acyl” denotes a C₁₋₆ alkyl radical attached to a carbonyl group wherein the definition of alkyl has the same definition as described herein; some examples include acetyl, propionyl, butanoyl, iso-butanoyl, pentanoyl, hexanoyl, heptanoyl, and the like.
The term “C₁₋₆ acyloxy” denotes an acyl radical attached to an oxygen atom wherein acyl has the same definition has described herein; some examples include acetyloxy, propionyloxy, butanoyloxy, iso-butanoyloxy and the like.
The term “C₂₋₆ alkenyl” denotes a radical containing 2 to 6 carbons, some embodiments are 2 to 4 carbons, some embodiments are 2 to 3 carbons, and some embodiments have 2 carbons. Both E and Z isomers are embraced by the term “alkenyl.” Furthermore, the term “alkenyl” includes di- and tri-alkenyls. Accordingly, if more than one double bond is present then the bonds may be all E or Z or a mixtures of E and Z. Examples of an alkenyl include vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl, 2,4-hexadienyl and the like.
The term “C₁₋₆ alkoxy” as used herein denotes a radical alkyl, as defined herein, attached directly to an oxygen such as methoxy, ethoxy, n-propoxy, iso-propoxy, n-butoxy, t-butoxy, iso-butoxy, sec-butoxy and the like.
The term “alkyl” denotes a radical containing 1 to 8 carbons, some embodiments are 1 to 6 carbons, some embodiments are 1 to 4 carbons, some embodiments are 1 to 3 carbons, and some embodiments are 1 or 2 carbons. Examples of an alkyl include methyl, ethyl, n-propyl, iso-propyl, n-butyl, sec-butyl, 2-butyl, t-butyl, amyl, t-amyl, 1-pentyl, 2,2-dimethyl-propyl, 3-pentyl, 3-methyl-butyl, 1,3-dimethyl-butyl, 3,3-dimethyl-butyl, hexyl, 3-methyl-butyl, 4-methyl-pentyl, 1-heptyl, and the like.
The term “C₁₋₆ alkylcarboxamido” denotes a single alkyl group attached to the amine of an amide, wherein alkyl has the same definition as found herein. Examples include N-methylcarboxamide, N-ethylcarboxamide, N-(iso-propyl)carboxamide and the like.
The term “C₁₋₆ alkynyl” denotes a radical containing 2 to 6 carbons, some embodiments are 2 to 4 carbons, some embodiments are 2 to 3 carbons, and some embodiments have 2 carbons. Examples of an alkynyl include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.
The term “C₁₋₆ alkylsulfinyl” denotes an alkyl radical attached to a sulfoxide radical of the formula: —S(O)— wherein the alkyl radical has the same definition as described herein. Examples include methylsulfinyl, ethylsulfinyl and the like.
The term “C₁₋₆ alkylsulfonyl” denotes an alkyl radical attached to a sulfone radical of the formula: —S(O)₂— wherein the alkyl radical has the same definition as described herein. Examples include methylsulfonyl, ethylsulfonyl and the like.
The term “C₁₋₆ alkylthio” denotes an alkyl radical attached to a sulfide of the formula: —S— wherein the alkyl radical has the same definition as described herein. Examples include methylsulfanyl (i.e., CH₃S—), ethylsulfanyl, isopropylsulfanyl and the like.
The term “C₁₋₆ alkylureyl” denotes the group of the formula: —NC(O)N— wherein one are both of the nitrogens are substituted with the same or different alkyl group wherein alkyl has the same definition as described herein. Examples of an alkylureyl include, CH₃NHC(O)NH—, NH₂C(O)NCH₃—, (CH₃)₂N(O)NH—, (CH₃)₂N(O)NH—, (CH₃)₂N(O)NCH₃—, CH₃CH₂NHC(O)NH—, CH₃CH₂NHC(O)NCH₃—, and the like.
The term “amino” denotes the group —NH₂.
The term “C₁₋₆ alkylamino” denotes an alkyl radical attached to an amino radical wherein the alkyl radical has the same meaning as described herein.
The term “C₁₋₆ dialkylamino” denotes an amino substituted with two of the same or different alkyl radicals wherein alkyl radical has the same definition as described herein. A C₁ dialkylamino may be represented by the following groups: 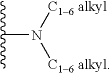
Examples of C₁₋₆ dialkylamino include, but not limited to, dimethylamino, methylethylamino, diethylamino, methylpropylamino, methylisopropylamino, and the like. Some examples include dimethylamino, methylethylamino, diethylamino and the like.
The term “aryl” denotes an aromatic ring radical containing 6 to 10 ring carbons, for example phenyl, napthyl and the like.
The term “benzyl” denotes the group —CH₂C₆H₅.
The term “carbo-C₁alkoxy” refers to an alkyl ester of a carboxylic acid, wherein the alkyl group is C¹⁻³. Examples include carbomethoxy, carboethoxy, carboisopropoxy and the like.
The term “carboxy” or “carboxyl” denotes the group —O₂H; also referred to as a carboxylic acid.
The term “cyano” denotes the group —CN.
The term “C₃₋₆cycloalkyl” denotes a saturated ring radical containing 3 to 6 carbons, some embodiments contain 3 to 5 carbons, and some embodiments contain 3 to 4 carbons. Examples include cyclopropyl, cyclobutyl, cyclopentyl, cyclohexyl and the like.
The term “C₁₋₆ dialkylcarboxanido” denotes two alkyl radicals, that are the same or different, attached to the amine of an amide, wherein alkyl has the same definition as described herein. Examples of a dialkylcarboxamide include N,N-dimethylcarboxamide, N-methyl-N-ethylcarboxamide and the like.
The term “halo” or “halogen” denotes to a fluoro, chloro, bromo or iodo group.
The term “C₁₋₆ haloalkoxy” denotes a haloalkyl, as defined herein, that is directly attached to an oxygen to form a difluoromethoxy, trifluoromethoxy, 2,2,2-trifluoroethoxy, pentafluoroethoxy and the like.
The term “C₁₋₆ haloalkyl” denotes an alkyl group, defined herein, wherein the alkyl is substituted with one halogen up to fully substituted represented by the formula CₙF₂ₙ₊₁; when more than one halogen is present they may be the same or different and selected from F, Cl, Br or I. Examples include fluoromethyl, difluoromethyl, trifluoromethyl, chlorodifluoromethyl, 2,2,2-trifluoroethyl, pentafluoroethyl and the like.
The term “C₁₋₆ haloalkylsulfinyl” denotes a haloalkyl radical attached to a sulfoxide of the formula: —S(O)— wherein the alkyl radical has the same definition as described herein. Examples include trifluoromethylsulfinyl, 2,2,2-trifluoroethylsulfinyl, 2,2-difluoroethylsulfinyl and the like.
The term “C₁₋₆ haloalkylsulfonyl” denotes a haloalkyl attached to a sulfone of the formula: —S(O)₂— wherein haloalkyl has the same definition as described herein. Examples include trifluoromethylsulfonyl, 2,2,2-trifluoroethylsulfonyl, 2,2-difluoroethylsulfonyl and the like.
The term “C₁₋₆ haloalkylthio” denotes an alkylthio radical substituted with one or more halogens. Examples include trifluoromethylthio, 1,1-difluoroethylthio, 2,2,2-trifluoroethylthio and the like.
The term “heteroaryl” denotes an aryl ring wherein one or more of the ring carbons are replaced by a ring nitrogen, examples include, pyridyl, pyrazinyl, pyridazinyl, pyrimidinyl, triazinyl, and the like.
The term “heterocyclyl” denotes a non-aromatic carbon ring (i.e., cycloalkyl) where one, two or three ring carbons are independently replaced with a heteroatom, such as, piperidinyl, morpholinyl, piperzinyl, pyrrolidinyl, tetrahydrofuranyl and the like.
The term “hydroxyl” denotes the group —OH.
The term “nitro” denotes the group —NO₂.
The term “perfluoroalkyl” denotes the group of the formula —CₙF₂ₙ₊₁; stated differently, a perfluoroalkyl is an alkyl as defined herein wherein the alkyl is fully substituted with fluorine atoms and is therefore considered a subset of haloalkyl. Examples of perfluoroalkyls include CF₃, CF₂CF₃, CF₂CF₂CF₃, CF(CF₃)₂, CF₂CF₂CF₂CF₃, CF₂CF(CF₃)₂, CF(CF₃)CF₂CF₃ and the like.
The term “thiol” denotes the group —SH.
The term “substituted aryl” denotes an aryl group as defined herein that is substituted with 1, 2, 3, 4, or 5 substituents selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro and thiol. Examples of a substituted aryl include, but not limited to, 3-methoxyphenyl, 4-methoxyphenyl, 3,5-difluorophenyl, and the like.

COMPOSITION means a material comprising at least one component; a “pharmaceutical composition” is an example of a composition.

COMPOUND EFFICACY shall mean a measurement of the ability of a compound to inhibit or stimulate receptor functionality; i.e. the ability to activate/inhibit a signal transduction pathway, in contrast to receptor binding affinity. Exemplary means of detecting compound efficacy are disclosed in the Example section of this patent document.

CONTACT or CONTACTING shall mean bringing at least two moieties together, whether in an in vitro system or an in vivo system. Thus, “contacting” a RUP38 receptor with a compound of the invention includes the administration of a compound of the present invention to an individual, for example a human, having a RUP38 receptor, as well as, for example, introducing a compound of the invention into a sample containing a cellular or more purified preparation containing a RUP38 receptor.

CORONARY HEART DISEASE is intended herein to encompass disorders comprising a narrowing of the small blood vessels that supply blood and oxygen to the heart. CORONARY HEART DISEASE usually results from the build up of fatty material and plaque. As the coronary arteries narrow, the flow of blood to the heart can slow or stop. CORONARY HEART DISEASE can cause chest pain (stable angina), shortness of breath, heart attack, or other symptoms.

DECREASE is used to refer to a reduction in a measurable quantity and is used synonymously with the terms “reduce”, “diminish”, “lower”, and “lessen”.

DIABETES as used herein is intended to encompass the usual diagnosis of DIABETES made from any of the methods including, but not limited to, the following list: symptoms of diabetes (e.g., polyuria, polydipsia, polyphagia) plus casual plasma glucose levels of greater than or equal to 200 mg/dl, wherein casual plasma glucose is defined any time of the day regardless of the timing of meal or drink consumption; 8 hour fasting plasma glucose levels of less than or equal to 126 mg/dl; and plasma glucose levels of greater than or equal to 200 mg/dl 2 hours following oral administration of 75 g anhydrous glucose dissolved in water.

DISORDERS OF LIPID METABOLISM is intended herein to include, but not be limited to, dyslipidemia.

DYSLIPIDEMIA is intended herein to encompass disorders comprising any one of elevated level of plasma free fatty acids, elevated level of plasma cholesterol, elevated level of LDL-cholesterol, reduced level of HDL-cholesterol, and elevated level of plasma triglycerides.

The term HYDRATE OR SOLVATE THEREOF as used herein and in the claims is intended to include hydrated forms such as monohydrate, dihydrate, hemihydrate, sesquihydrate, trihydrate, tetrahydrate and the like as well as solvated forms. The products may be true hydrates, while in other cases, the products may merely retain adventitious water or be a mixture of water plus some adventitious solvent. It should be appreciated by those skilled in the art that hydrated and/or solvated forms are equivalent to unsolvated forms and are intended to be encompassed within the scope of the present invention.

The phrase IN NEED OF TREATMENT, as used herein, refers to a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, that includes the knowledge that the individual is ill, or will be ill, as the result of a disease, condition or disorder that is treatable by the compounds of the invention. Further, the phrase “in need of treatment” also refers to the “prophylaxis” of an individual which is the judgment made by the caregiver that the individual will become ill. In this context, the compounds of the invention are used in a protective or preventive manner. Accordingly, “in need of treatment” refers to the judgment of the caregiver that the individual is already ill or will become ill and the compounds of the present invention can be used to alleviate, inhibit, ameliorate or prevent the disease, condition or disorder.

INDIRECTLY IDENTIFYING or INDIRECTLY IDENTIFIED means the traditional approach to the drug discovery process involving identification of an endogenous ligand specific for an endogenous receptor, screening of candidate compounds against the receptor for determination of those which interfere and/or compete with the ligand-receptor interaction, and assessing the efficacy of the compound for affecting at least one second messenger pathway associated with the activated receptor.

INDIVIDUAL as used herein refers to any animal, including mammals, preferably mice, rats, other rodents, rabbits, dogs, cats, swine, cattle, sheep, horses, or primates, and most preferably humans.

INHIBIT or INHIBITING, in relationship to the term “response” shall mean that a response is decreased or prevented in the presence of a compound as opposed to in the absence of the compound.

INSULIN RESISTANCE as used herein is intended to encompass the usual diagnosis of insulin resistance made by any of a number of methods, including but not restricted to: the intravenous glucose tolerance test or measurement of the fasting insulin level. It is well known that there is an excellent correlation between the height of the fasting insulin level and the degree of insulin resistance. Therefore, one could use elevated fasting insulin levels as a surrogate marker for insulin resistance for the purpose of identifying which normal glucose tolerance (NGT) individuals have insulin resistance. A diagnosis of insulin resistance can also be made using the euglycemic glucose clamp test.

The term INVERSE AGONISTS shall mean moieties that bind the endogenous form of the receptor or to the constitutively activated form of the receptor, and which inhibit the baseline intracellular response initiated by the active form of the receptor below the normal base level of activity which is observed in the absence of agonists or partial agonists, or decrease GTP binding to membranes. In some embodiments, the baseline intracellular response is inhibited in the presence of the inverse agonist by at least 30%, in other embodiments, by at least 50%, and in still other embodiments, by at least 75%, as compared with the baseline response in the absence of the inverse agonist.

LIGAND shall mean a molecule specific for a naturally occurring receptor.

METABOLIC-RELATED DISORDERS are intended herein to include, but not be limited to, dyslipidemia, atherosclerosis, coronary heart disease, insulin resistance, obesity, impaired glucose tolerance, atheromatous disease, hypertension, stroke, Syndrome X, heart disease and type 2 diabetes.

As used herein, the terms MODULATE or MODULATING shall mean to refer to an increase or decrease in the amount, quality, response or effect of a particular activity, function or molecule.

PARTIAL AGONISTS shall mean materials (e.g., ligands, candidate compounds) that activate the intracellular response when they bind to the receptor to a lesser degree/extent than do full agonists.

PHARMACEUTICAL COMPOSITION shall mean a composition comprising at least one active ingredient whereby the composition is amenable to investigation for a specified, efficacious outcome in a mammal (for example, and not limitation, a human). Those of ordinary skill in the art will understand and appreciate the techniques appropriate for determining whether an active ingredient has a desired efficacious outcome based upon the needs of the artisan.

The term PHARMACEUTICALLY ACCEPTABLE CARRIER or EXCIPIENT shall mean any substantially inert substance used as a diluent or vehicle for a compound of the present invention.

The phrase THERAPEUTICALLY EFFECTIVE AMOUNT as used herein refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) Preventing the disease; for example, preventing a disease, condition or disorder in an individual that may be predisposed to the disease, condition or disorder but does not yet experience or display the pathology or symptomatology of the disease,

(2) Inhibiting the disease; for example, inhibiting a disease, condition or disorder in an individual that is experiencing or displaying the pathology; or symptomatology of the disease, condition or disorder (i.e., arresting further development of the pathology and/or symptomatology), and

(3) Ameliorating the disease; for example, ameliorating a disease, condition or disorder in an individual that is experiencing or displaying the pathology or symptomatology of the disease, condition or disorder (i.e., reversing the pathology and/or symptomatology).

Synthetic Methods of Benzotriazoles

The compounds of the present invention can be readily prepared according to a variety of synthetic regimes, all of which would be familiar to one skilled in the art. The chemical and patent literature quotes general procedures for the synthesis of benzotriazoles. Some relevant references include: James, D. R. and Felix, R. A., PCT Int. Application WO9425446 A1; Katritzky A. R. and Rees, C. W., Comprehensive Heterocyclic Chemistry, Pergamon Press, 1996.

In the illustrated syntheses outlined below, the labeled substituents have the same identifications as set out in the definitions of the compound described above. As shown below, the methods described thereafter may be used for the preparation of compound of Formula (I).

The benzotriazoles derivatives of the Formula (I) of the present invention may be prepared by the following exemplary general procedure as described in Reaction Scheme (1) shown below: 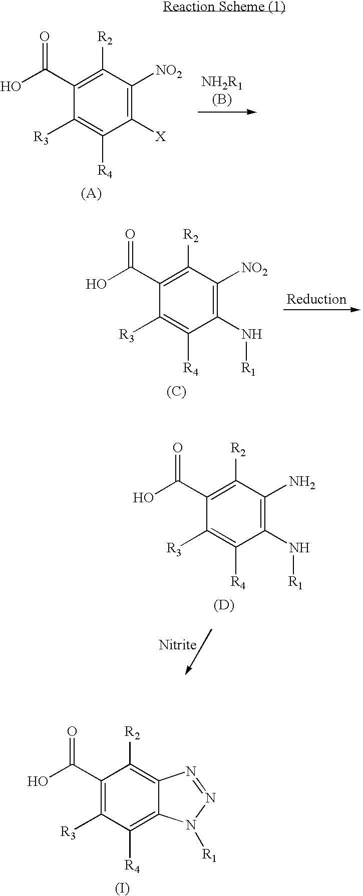
In this instance, the R₁ group is introduced via a displacement reaction of an ortho-halo nitrobenzene (A) with amine (B). A variety of amines can be purchased or prepared by methods known in the art and therefore a diverse set of R₁ groups may be introduced, see below for further discussion. Intermediate (C) can be converted to diamino (D) by a variety of reducing methods, such as, tin under acidic conditions, alcoholic ammonium sulfide with heat, hydrogen in the presence of Pd/C, iron or SnCl₂. The resulting ortho phenylenediamine can be readily cyclized to the compounds of Formula (I) by treatment with nitrite, such as NaNO₂ or isoamyl nitrite in the presence of an acid.

As mentioned, a variety of R₁ may be introduced into compounds of the present invention via an appropriate amine. A variety of these amines are commercially available or readily prepared by methods known in the art. For example, R₁ may be a haloalkyl, some exemplary haloalkyl amines include, 1,1,1,3,3,3-hexafluoro-2-amino-propane and 1,1,1,2,3,3,3-heptafluoro-2-amino-propane and can be prepared from the readily available hexafluoroacetone by the methods described by Middleton and co-workers in J. Org. Chem., 1965, 30, 1398-1402. Other amines include 2,2,2-trifluoroethylamine, 3,3,3,2,2-pentafluoropropylamine, 3,3,3-trifluoropropylamine, and the like. Similarly, R₁ may be a cycloalkyl and in accordance with Reaction Scheme (I) a number of cycloalkyl groups may be introduced using this method. For example, cyclopropyl amine, cyclobutyl amine, cyclopentyl amine and cyclohexyl amine may be utilized to afford compounds of Formula (I). In the example where R₁ is a cyclopropyl group an analogous displacement step in Reaction Scheme (I) has been reported in the literature by Cecchetti, A. and co-workers in J. Med. Chem. 1995, 38, 973-982; a similar reaction has also been reported for cyclopentyl amine by Pan, P-C and Sun, C-M in Bioorg, Med. Chem. Lett. 1999, 9, 1537-1540. In addition, a variety of substituted cycloalkyl amines are commercially available or may be prepared by methods known in the art, for example, a variety of cyclopropyl amines may be prepared from a nitrile and a Grignard reagent in the presence of a reagent such as, Ti(i-OPr)₄, and followed by treatment with BF₃.Et₂O (Bertus, P. and Szymoniak, J. in Chem. Comm. 2001, 18, 1792-1793). Other methods are known for the preparation of cycloalkyl amines and substituted cycloalkyl amines.

An alternative method for the preparation of compounds of Formula (I) is shown in Reaction Scheme (2): 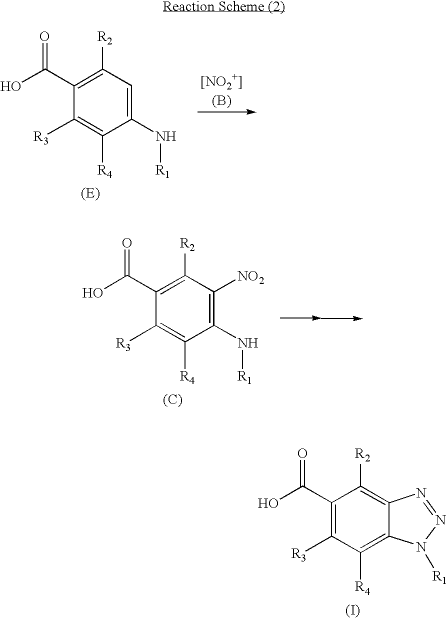
This method may utilize a variety of anilines as starting materials. These anilines may be converted into intermediate CE) by methods known in the art, such as, alkylation, reductive amination and the like. Subsequently, intermediate (E) may be nitrated to give intermediate (C) and the remaining steps in Reaction Scheme (2) are similar to those described above in Reaction Scheme (1).

The various organic group transformations utilized herein may be performed through a number of procedures other than those described above. References for other synthetic procedures that may be utilized for the preparation of intermediates or compounds disclosed herein may be found in, for example, Smith, M. B.; and March, J., Advanced Organic Chemistry, 5^{th} Edition, Wiley-Interscience (2001); Larock, R. C., Comprehensive Organic Transformations, A Guide to Functional Group Preparations, 2^{nd} Edition, VCH Publishers, Inc. (1999), or Wuts, P. G. M.; Greene, T. W.; Protective Groups in Organic Synthesis, 3^{rd} Edition, John Wiley and Sons, (1999), all three incorporated herein by reference.

Representative examples are shown below in Tables B and C.

TABLE B
Wherein R₁ is a cyclopropyl (i.e., cC₃H₅—) or cyclobutyl (cC₄H₇—)
radical

| | |
|---|---|
| | (I) |
| 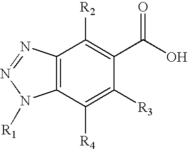 | |

| R₁ | R₂ | R₃ | R₄ |
|---|---|---|---|
| cC₃H₅— | H | H | H |
| cC₃H₅— | H | H | F |
| cC₃H₅— | H | F | H |
| cC₃H₅— | F | H | H |
| cC₃H₅— | H | F | F |
| cC₃H₅— | F | H | F |
| cC₃H₅— | F | F | H |
| cC₃H₅— | F | F | F |
| cC₃H₅— | H | H | Cl |
| cC₃H₅— | H | Cl | H |
| cC₃H₅— | Cl | H | H |
| cC₄H₇— | H | H | H |
| cC₄H₇— | H | H | F |
| cC₄H₇— | H | F | H |
| cC₄H₇— | F | H | H |
| cC₄H₇— | H | F | F |
| cC₄H₇— | F | H | F |
| cC₄H₇— | F | F | H |
| cC₄H₇— | F | F | F |
| cC₄H₇— | H | H | Cl |
| cC₄H₇— | H | Cl | H |
| cC₄H₇— | Cl | H | H |

TABLE C
Where R₁ is a 2,2,2-trifluoroethyl or 1-(2,2,2-Trifluoro-1-trifluoromethyl-
ethyl) group

| | |
|---|---|
| | (I) |
| 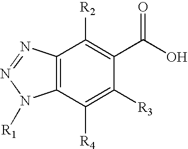 | |

| R₁ | R₂ | R₃ | R₄ |
|---|---|---|---|
| CF₃CH₂— | H | H | H |
| CF₃CH₂— | H | H | F |
| CF₃CH₂— | H | F | H |
| CF₃CH₂— | F | H | H |
| CF₃CH₂— | H | F | F |
| CF₃CH₂— | F | H | F |
| CF₃CH₂— | F | F | H |
| CF₃CH₂— | F | F | F |
| CF₃CH₂— | H | H | Cl |
| CF₃CH₂— | H | Cl | H |
| CF₃CH₂— | Cl | H | H |
| (CF₃)₂CH— | H | H | H |
| (CF₃)₂CH— | H | F | H |
| (CF₃)₂CH— | F | H | H |
| (CF₃)₂CH— | H | F | F |
| (CF₃)₂CH— | F | H | F |
| (CF₃)₂CH— | F | F | H |
| (CF₃)₂CH— | F | F | F |
| (CF₃)₂CH— | H | H | Cl |
| (CF₃)₂CH— | H | Cl | H |
| (CF₃)₂CH— | Cl | H | H |

Additionally, compounds of Formula (I) encompass all pharmaceutically acceptable solvates, particularly hydrates, thereof. The present invention also encompasses diastereomers as well as optical isomers, e.g. mixtures of enantiomers including racemic mixtures, as well as individual enantiomers and diastereomers, which arise as a consequence of structural asymmetry in certain compounds of Formula (I). Separation of the individual isomers or selective synthesis of the individual isomers is accomplished by application of various methods which are well known to practitioners in the art.

Pharmaceutical Compositions

A compound of the present invention can be formulated into pharmaceutical compositions using techniques well known to those in the art. Suitable pharmaceutically-acceptable carriers, outside those mentioned herein, are available to those in the art; for example, see Remington's Pharmaceutical Sciences, 16^{th} Edition, 1980, Mack Publishing Co., (Oslo et al., eds.) or a more recent edition thereof.

While it is possible that, for use in the prophylaxis or treatment, a compound of the invention may in an alternative use be administered as a raw or pure chemical, it is preferable however to present the compound or active ingredient as a pharmaceutical formulation or composition further comprising a pharmaceutically acceptable carrier.

The invention thus further provides pharmaceutical formulations comprising a compound of the invention or a pharmaceutically acceptable salt or derivative thereof together with one or more pharmaceutically acceptable carriers thereof and/or prophylactic ingredients. The carrier(s) must be “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation.

The compounds of the invention, together with a conventional adjuvant, carrier, or diluent, may thus be placed into the form of pharmaceutical formulations and unit dosages thereof, and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable therapeutically effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

The dose when using the compounds of Formula (I) can vary within wide limits, and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds of the Formula (I). Representative doses of the present invention include, about 0.01 mg to about 1000 mg, about 0.01 to about 750 mg, about 0.01 to about 500 mg, 0.01 to about 250 mg, 0.01 mg to about 200 mg, about 0.01 mg to 150 mg, about 0.01 mg to about 100 mg, and about 0.01 mg to about 75 mg. Multiple doses may be administered during the day, especially when relatively large amounts are deemed to be needed, for example 2, 3 or 4, doses. If appropriate, depending on individual behavior and as appropriate from the patients physician or care-giver it may be necessary to deviate upward or downward from the daily dose.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. Typically, animal models include, but are not limited to, the rodent diabetes models as described in Example 1, infra, or the mouse arthrosclerosis model as described in Example 2, infra. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacolinetic and toxicology profiles of the particular compound employed, whether a drug delivery system is utilized, on whether an acute or chronic disease state is being treated or prophylaxis is conducted or on whether further active compounds are administered in addition to the compounds of the Formula (I) and as part of a drug combination. The dosage regimen for treating a disease condition with the compounds and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4, part administrations. If appropriate, depending on individual behavior, it may be necessary to deviate upward or downward from the daily dose indicated.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt of a compound of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size.

The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms, as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the Formula (I) or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the Formula (I) as an aerosol can be prepared by processes well-known to the person skilled in the art. For their preparation, for example, solutions or dispersions of the compounds of the Formula (I) in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example include carbon dioxide, CFC's, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The term “prodrug” refers to compounds that are rapidly transformed in vivo to yield the parent compound of the above formulae, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference.

Combination Therapy/Prophylaxis

While the compounds of the invention can be administered as the sole active pharmaceutical agent as described herein above, they can also be used in combination with one or more agents belonging to the class of drugs known as α-glucosidase inhibitors, aldose reductase inhibitors, biguanides, IBG-CoA reductase inhibitors, squalene synthesis inhibitors, fibrate compounds, LDL catabolism enhancers and angiotensin converting enzyme (ACE) inhibitors.

α-Glucosidase inhibitors belong to the class of drugs which competitively inhibit digestive enzymes such as α-amylase, maltase, α-dextrinase, sucrase, etc. in the pancreas and or small intestine. The reversible inhibition by α-glucosidase inhibitors retard, diminish or otherwise reduce blood glucose levels by delaying the digestion of starch and sugars. Some representative examples of α-glucosidase inhibitors include acarbose, N-(1,3-dihydroxy-2-propyl)valiolamine (generic name; voglibose), miglitol, and α-glucosidase inhibitors known in the art.

The class of aldose reductase inhibitors are drugs which inhibit the first-stage rate-limiting enzyme in the polyol pathway that prevent or arrest diabetic complications. In the hyperglycemic state of diabetes, the utilization of glucose in the polyol pathway is increased and the excess sorbitol accumulated intracellularly as a consequence acts as a tissue toxin and hence evokes the onset of complications such as diabetic neuropathy, retinopathy, and nephropathy. Examples of the aldose reductase inhibitors include tolurestat; epalrestat; 3,4-dihydro-2,8-diisopropyl-3-thioxo-2H-1,4-benzoxazine-4-acetic acid; 2,7-difluorospiro(9H-fluorene-9,4′-imidazolidine)-2′,5′-dione (generic name: imirestat); 3-[(4-bromo-2-flurophenyl)methy]-7-chloro-3,4-dihydro-2,4-dioxo-[(2H)-quinazoline acetic acid (generic name: zenarestat); 6-fluoro-2,3-dihydro-2′,5′-dioxo-spiro[4H-1-benzopyran-4,4′-imidazolidine]-2-carboxamide (SNK-860); zopolrestat; sorbinil; and 1-[(3-bromo-2-benzofuranyl)sulfonyl]-2,4-imidazolidinedione (M-16209), and aldose reductase inhibitors known in the art.

The biguanides are a class of drugs that stimulate anaerobic glycolysis, increase the sensitivity to insulin in the peripheral tissues, inhibit glucose absorption from the intestine, suppress of hepatic gluconeogenesis, and inhibit fatty acid oxidation. Examples of biguanides include phenformin, metformin, buformin, and biguanides known in the art.

Statin compounds belong to a class of drugs that lower blood cholesterol levels by inhibiting hydroxymethylglutalyl CoA (HMG-CoA) reductase. HMG-CoA reductase is the rate-limiting enzyme in cholesterol biosynthesis. A statin that inhibits this reductase lowers serum LDL concentrations by upregulating the activity of LDL receptors and responsible for clearing LDL from the blood. Examples of the statin compounds include rosuvastatin, pravastatin and its sodium salt, simvastatin, lovastatin, atorvastatin, fluvastatin, cerivastatin, and HMG-CoA reductase inhibitors known in the art.

Squalene synthesis inhibitors belong to a class of drugs that lower blood cholesterol levels by inhibiting synthesis of squalene. Examples of the squalene synthesis inhibitors include (S)-α-[Bis[2,2-dimethyl-1-oxopropoxy)methoxy]phosphinyl]-3-phenoxybenzenebutanesulfonic acid, mono potassium salt BMS-188494) and squalene synthesis inhibitors known in the art.

Fibrate compounds belong to a class of drugs that lower blood cholesterol levels by inhibiting synthesis and secretion of triglycerides in the liver and activating a lipoprotein lipase. Fibrates have been known to activate peroxisome proliferators-activated receptors and induce lipoprotein lipase expression. Examples of fibrate compounds include bezafibrate, beclobrate, binifibrate, ciplofibrate, clinofibrate, clofibrate, clofibric acid, etofibrate, fenofibrate, gemfibrozil, nicofibrate, pirifibrate, ronifibrate, simfibrate, theofibrate, and fibrates known in the art.

LDL (low-density lipoprotein) catabolism enhancers belong to a class of drugs that lower blood cholesterol levels by increasing the number of LDL (low-density lipoprotein) receptors, examples include LDL catabolism enhancers known in the art.

Angiotensin converting enzyme (ACE) inhibitors belong to the class of drugs that partially lower blood glucose levels as well as lowering blood pressure by inhibiting angiotensin converting enzymes. Examples of the angiotensin converting enzyme inhibitors include captopril, enalapril, alacepril, delapril; raripril, lisinopril, imidapril, benazepril, ceronapril, cilazapril, enalaprilat, fosinopril, moveltopril, perindopril, quinapril, spirapril, temocapril, trandolapril, and angiotensin converting enzyme inhibitors known in the art.

Insulin secretion enhancers belong to the class of drugs having the property to promote secretion of insulin from pancreatic β cells. Examples of the insulin secretion enhancers include sulfonylureas (SU). The sulfonylureas (SU) are drugs which promote secretion of insulin from pancreatic β cells by transmitting signals of insulin secretion via SU receptors in the cell membranes. Examples of the sulfonylureas include tolbutamide; chlorpropamide; tolazamide; acetohexamide; 4-chloro-N-[(1-pyrolidinylamino)carbonyl]-benzenesulfonamide (generic name: glycopyramide) or its ammonium salt; glibenclamide (glyburide); gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolcyclamide, glimepiride, and other insulin secretion enhancers known in the art. Other insulin secretion enhancers include N-[[4-(1-methylethyl)cyclohexyl)carbonyl]-D-phenylalanine (Nateglinide); calcium (2S)-2-benzyl-3-(cis-hexahydro-2-isoindolinylcarbonyl)propionate dihydrate (Mitiglinide, KAD-1229); and other insulin secretion enhancers known in the art.

Thiazolidinediones belong to the class of drugs more commoningly known as TZDs. Examples of thiazolidinediones include rosiglitazone, pioglitazone, and thiazolidinediones known in the art.

Some embodiments of the invention include, a pharmaceutical composition comprising a compound of Formula (I) or a pharmaceutically acceptable salt thereof in combination with at least one member selected from the group consisting of an α-glucosidase inhibitor, an aldose reductase inhibitor, a biguanide, a HMG-CoA reductase inhibitor, a squalene synthesis inhibitor, a fibrate compound, a LDL catabolism enhancer and an angiotensin converting enzyme inhibitor. In another embodiment, the pharmaceutical composition is a compound of Formula (I) or a pharmaceutically acceptable salt thereof in combination with a HMG-CoA reductase inhibitor. In still another embodiment, the HMG-CoA reductase inhibitor is selected from the group consisting of prevastatin, simvastatin, lovastatin, atorvastatin, fluvastatin and lipitor.

In accordance with the present invention, the combination can be used by mixing the respective active components either all together or independently with a physiologically acceptable carrier, excipient, binder, diluent, etc., as described herein above, and administering the mixture or mixtures either orally or non-orally as a pharmaceutical composition. When a compound or a mixture of compounds of Formula (I) are administered as a combination therapy or prophylaxis with another active compound the therapeutic agents can be formulated as a separate pharmaceutical compositions given at the same time or at different times, or the therapeutic agents can be given as a single composition.

Labeled Compounds and Assay Methods

Another object of the present invention relates to radio-labeled compounds of Formula (1) that are useful not only in radio-imaging but also in assays, both in vitro and in vivo, for localizing and quantitating hRUP38 in tissue samples, including human, and for identifying hRUP38 ligands by inhibition binding of a radio-labeled compound. It is a further object of this invention to include novel hRUP38 assays of which comprise such radio-labeled compounds.

The present invention embraces isotopically-labeled compounds of Formula (I) and any subgenera herein, such as but not limited to, Formulae (Ia) to (Is). An “isotopically” or “radio-labeled” compounds are those which are identical to compounds disclosed herein, but for the fact that one or more atoms are replaced or substituted by an atom having an atomic mass or mass number different from the atomic mass or mass number typically found in nature (i.e., naturally occurring). Suitable radionuclides that can be incorporated in compounds of the present invention include but are not limited to ²H (also written as D for deuterium), ³H (also written as T for tritium), ¹¹C, ¹³C, ¹⁴C, ¹³N ¹⁵N, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro hRUP38 labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound of Formula (I) that has incorporated at least one radionuclide; in some embodiments the radionuclide is selected from the group consisting of ³H, ¹⁴C, ¹²⁵I, ³⁵S and ⁸²Br.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence can be preferred in some circumstances. Isotopically labeled compounds of the present invention can generally be prepared by following procedures analogous to those disclosed in the Schemes supra and Examples infra, by substituting an isotopically labeled reagent for a non-isotopically labeled reagent. Other synthetic methods that are useful are discussed infra. Moreover, it should be understood that all of the atoms represented in the compounds of the invention can be either the most commonly occurring isotope of such atoms or the scarcer radio-isotope or nonradio-active isotope.

Synthetic methods for incorporating radio-isotopes into organic compounds are applicable to compounds of the invention and are well known in the art. These synthetic methods, for example, incorporating activity levels of tritium into target molecules, and are as follows:

A. Catalytic Reduction with Tritium Gas—This procedure normally yields high specific activity products and requires halogenated or unsaturated precursors.

B. Reduction with Sodium Borohydride [³H]—This procedure is rather inexpensive and requires precursors containing reducible functional groups such as aldehydes, ketones, lactones, esters, and the like.

C. Reduction with Lithium Aluminum Hydride [³H]—This procedure offers products at almost theoretical specific activities. It also requires precursors containing reducible functional groups such as aldehydes, ketones, lactones, esters, and the like.

D. Tritium Gas Exposure Labeling—This procedure involves exposing precursors containing exchangeable protons to tritium gas in the presence of a suitable catalyst.

E. N-Methylation using Methyl Iodide [³H]—This procedure is usually employed to prepare O-methyl or N-methyl (³H products by treating appropriate precursors with high specific activity methyl iodide (³H). This method in general allows for higher specific activity, such as for example, about 70-90 Ci/mmol.

Synthetic methods for incorporating activity levels of ¹²⁵I into target molecules include:

A. Sandmeyer and like reactions—This procedure transforms an aryl or heteroaryl amine into a diazonium salt, such as a tetrafluoroborate salt, and subsequently to ¹²⁵I labeled compound using Na¹²⁵I. A represented procedure was reported by Zhu, D.-G. and co-workers in J. Org. Chem. 2002, 67, 943-948.

B. Ortho ¹²⁵Iodination of phenols—This procedure allows for the incorporation of ¹²⁵I at the ortho position of a phenol as reported by Collier, T. L. and co-workers in J. Labeled Compd Radiopharm. 1999, 42, S264-S266.

C. Aryl and heteroaryl bromide exchange with ¹²⁵I—This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A represented procedure was reported by Bas, M.-D. and co-workers in J. Labeled Compd Radiopharm. 2001, 44, S280-S282.

A radio-labeled hRUP38 compound of Formula (I) can be used in a screening assay to identify/evaluate compounds. In general terms, a newly synthesized or identified compound (i.e., test compound) can be evaluated for its ability to reduce binding of the “radio-labeled compound of Formula (I)” to the hRUP38 receptor. Accordingly, the ability of a test compound to compete with the “radio-labeled compound of Formula (I)” for the binding to the hRUP38 receptor directly correlates to its binding affinity.

The labeled compounds of the present invention bind to the hRUP38 receptor. In one embodiment the labeled compound has an IC₅₀ less than about 500 μM, in another embodiment the labeled compound has an IC₅₀ less than about 100 μM, in yet another embodiment the labeled compound has an IC₅₀ less than about 10 μM, in yet another embodiment the labeled compound has an IC₅₀ less than about 1 μM, and in still yet another embodiment the labeled inhibitor has an IC₅₀ less than about 0.1 μM.

Other uses of the disclosed receptors and methods will become apparent to those in the art based upon, inter alia, a review of this disclosure.

As will be recognized, the steps of the methods of the present invention need not be performed any particular number of times or in any particular sequence. Additional objects, advantages, and novel features of this invention will become apparent to those skilled in the art upon examination of the following examples thereof, which are intended to be illustrative and not intended to be limiting.

### EXAMPLES

The following examples are presented for purposes of elucidation, and not limitation, of the present invention. One of ordinary skill in the art would be able to design equivalent assays and methods based on the disclosure herein, all of which form part of the present invention.

### Example 1

Rodent Diabetes Models

Rodent models of type 2 diabetes associated with obesity and insulin resistance have been developed. Genetic models such as db/db and ob/ob [see Diabetes (1982) 31:1-6] in mice and fa/fa in zucker rats have been developed for understanding the pathophysiology of disease and for testing candidate therapeutic compounds [Diabetes (1983) 32:830-838; Annu Rep Sankyo Res Lab (1994) 46:1-57]. The homozygous animals, C57 BL/KsJ-db/db mice developed by Jackson Laboratory are obese, hyperglycemic, hyperinsulinemic and insulin resistant [J Clin Invest (1990) 85:962-967], whereas heterozygotes are lean and normoglycemic. In the db/db model, mice progressively develop insulinopenia with age, a feature commonly observed in late stages of human type 2 diabetes when sugar levels are insufficiently controlled. Since this model resembles that of human type 2 diabetes, the compounds of the present invention are tested for activities including, but not limited to, lowering of plasma glucose and triglycerides. Zucker (fa/fa) rats are severely obese, hyperinsulinemic, and insulin resistant {Coleman, Diabetes (1982) 31:1; E Shafrir in Diabetes Mellitus, H Rifkin and D Porte, Jr, Eds [Elsevier Science Publishing Co, New York, ed. 4, (1990), pp. 299-340]}, and the fa/fa mutation may be the rat equivalent of the murine db mutation [Friedman et al, Cell (1992) 69:217-220; Truett et al, Proc Natl Acad Sci USA (1991) 88:7806]. Tubby (tub/tub) mice are characterized by obesity, moderate insulin resistance and hyperinsulinemia without significant hyperglycemia [Coleman et al, Heredity (1990) 81:424).

The present invention encompasses the use of compounds of the invention for reducing the insulin resistance and hyperglycemia in any or all of the above rodent diabetes models, in humans with type 2 diabetes or other preferred metabolic-related disorders or disorders of lipid metabolism described previously, or in models based on other mammals. Plasma glucose and insulin levels will be tested, as well as other factors including, but not limited to, plasma free fatty acids and triglycerides.

In Vivo Assay for Anti-Hyperglycemic Activity of Compounds of the Invention

Genetically altered obese diabetic mice (db/db) (male, 7-9 weeks old) are housed (7-9 mice/cage) under standard laboratory conditions at 22° C. and 50% relative humidity, and maintained on a diet of Purina rodent chow and water ad libitum. Prior to treatment, blood is collected from the tail vein of each animal and blood glucose concentrations are determined using One Touch Basic Glucose Monitor System (Lifescan). Mice that have plasma glucose levels between 250 to 500 mg/dl are used. Each treatment group consists of seven mice that are distributed so that the mean glucose levels are equivalent in each group at the start of the study db/db mice are dosed by micro-osmotic pumps, inserted using isoflurane anesthesia, to provide compounds of the invention, saline, or an irrelevant compound to the mice subcutaneously (s.c.). Blood is sampled from the tail vein at intervals thereafter and analyzed for blood glucose concentrations. Significant differences between groups (comparing compounds of the invention to saline-treated) are evaluated using Student t-test.

### Example 2

Mouse Atherosclerosis Model

Adiponectin-deficient mice generated through knocking out the adiponectin gene have been shown to be predisposed to atherosclerosis and to be insulin resistant. The mice are also a suitable model for ischemic heart disease [Matsuda, M et al. J Biol Chem (2002) July, and references cited therein, the disclosures of which are incorporated herein by reference in their entirety].

Adiponectin knockout mice are housed (7-9 mice/cage) under standard laboratory conditions at 22° C. and 50% relative humidity. The mice are dosed by micro-osmotic pumps, inserted using isoflurane anesthesia, to provide compounds of the invention, saline, or an irrelevant compound to the mice subcutaneously (s.c.). Neointimal thickening and ischemic heart disease are determined for different groups of mice sacrificed at different time intervals. Significant differences between groups (comparing compounds of the invention to saline-treated) are evaluated using Student t-test.

### Example 3

Inhibition of Isoproterenol Stimulated Lipolysis in Human Subcutaneous Adipocytes

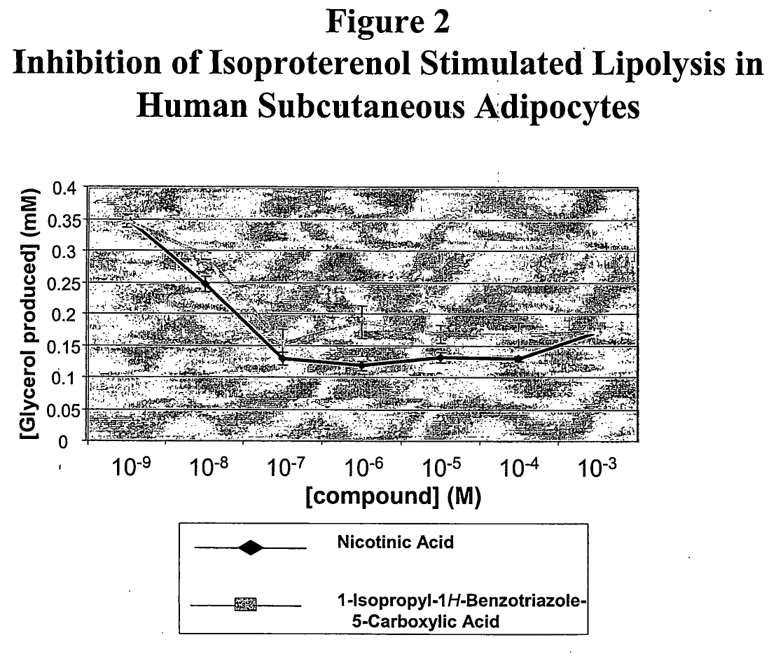

Nicotinic acid and 1-Isopropyl-1H-benzotriazole-5-carboxylic acid were separately dose-dependently applied to isoproterenol (100 nM) stimulated primary human adipocytes. FIG. 2 illustrates the ability of 1-Isopropyl-1H-benzotriazole-5-carboxylic acid to inhibit isoproterenol stimulated lipolysis in adipocyte primary cultures derived from human subcutaneous fat in a dose-dependent manner comparable to that of nicotinic acid.

### Example 4

In Vitro Biological Activity

A modified Flash Plate™ Adenylyl Cyclase kit (New England Nuclear; Cat. No. SMP004A) was utilized for direct identification of candidate compounds as agonists to hRUP38 (Seq. Id. Nos. 1 & 2) or hRUP25 (Seq. Id. Nos. 3 & 4) in accordance with the following protocol:

CHO cells stably transfected with hRUP38 were harvested from flasks via non-enzymatic means. The cells were washed in PBS and resuspended in the manufacturer's Assay Buffer. Live cells were counted using a hemacytometer and Trypan blue exclusion, and the cell concentration was adjusted to 2×10⁶ cells/ml. cAMP standards and Detection Buffer (comprising 2 μCi of tracer [¹²⁵I]-cAMP (100 μl) to 11 ml Detection Buffer) were prepared and maintained in accordance with the manufacturer's instructions. Candidate compounds identified as per above (if frozen, thawed at room temperature) were added to their respective wells (preferably wells of a 96-well plate) at increasing concentrations (3 μl/well; 12 μM final assay concentration). To these wells, 100,000 cells in 50 μl of Assay Buffer were added and the mixture was then incubated for 30 minutes at room temperature, with gentle shaking. Following the incubation, 100 μl of Detection Buffer was added to each well, followed by incubation for 2-24 hours. Plates were counted in a Wallac MicroBeta™ plate reader using “Prot. #31” (as per manufacturer instructions).

### Example 5

### Representative Biological Activity

The biological in vitro activity was determined using the cAMP Whole Cell method, one representative example is shown in the table below:

| | hRUP38 (EC₅₀) |
|---|---|
| Compound | cAMP Whole Cell (nM) |
| Example 6.1 | 388* |

*Value is an average of seven (7) trials.

Certain compounds in the Examples showed below display EC₅₀ activities in the cAMP Whole Cell (nM) assay of less than about 25 μM.

### Example 6.1

### Preparation of 1-Isopropyl-1H-benzotriazole-5-carboxylic acid

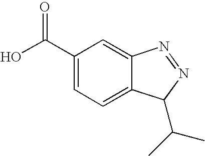

4-Isopropylamino-3-nitro-benzoic acid (0.077 g, 0.34 mmol) was taken up in ethyl acetate (30 mL), palladium (10% on carbon, 0.010 g) added and the suspension shaken at room temperature under a hydrogen atmosphere (balloon pressure) for 3 hours. The resulting solution was filtered through celite and solvent removed under reduced pressure to give 3-amino-4-ethylamino-benzoic acid as a pale brown glass. The diamine was taken up immediately in glacial acetic acid (5 mL), and polymer supported nitrate (0.030 g, loading ca 4 mmolg⁻¹, 0.12 mmol) added. The mixture was shaken overnight at room temperature under argon, filtered and solvent removed under reduced pressure to give 1-isopropyl-1H-benzotriazole-5-carboxylic acid as a brown crystalline solid (0.057 g, 0.28 mmol, 81%). m/z (ES⁺): 206 [M+H]⁺. ¹H NMR (CD₃OD): 8.58 (s, 1H, C(4)-H), 8.09 (dd, 1H, J₁=8.8, J₂=1.4, C(6)-H), 7.79 (dd, 1H, J₁=8.8, J₂=0.5, C(7)-H), 5.15 (septet, 1H, J=6.7, CH(CH₃)₂), 1.63 (d, 6H, J=6.7, CH(CH₃)₂).

The intermediate 4-isopropylamino-3-nitro-benzoic acid was prepared in the following manner:

a. 4-Isopropylamino-3-nitro-benzoic acid

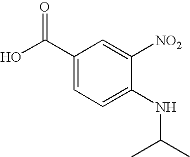

A mixture of 4-fluoro-3-nitrobenzoic acid (100 mg, 0.541 mmol), isopropyl amine (40 mg, 0.678) and sodium bicarbonate (0.10 g, 1.2 mmol) in H₂O (3 mL) was heated to 150° C. for 20 minutes under microwave irradiation. The resulting orange mixture was cooled, poured into 1 N HCl (40 mL) and extracted into EtOAc. The solvent was removed under reduced pressure to (4-isopropylamino-3-nitro-benzoic acid as a yellow solid which was used without further purification. ¹H NMR (CDCl₃) δ 9.05 (d, J=2.0 Hz, 1H), 8.47, (d, J=6.5 Hz, 1H), 8.16 (dd, J₁=9.1 Hz, J₂=2.0 Hz, 1H), 7.00 (d, J=9.1 Hz, 1H), 4.01 (septet, J=6.5 Hz, 1H), 1.47 (d, J=6.5 Hz, 6H).

### Example 6.2

### Preparation of 1-Cyclopentyl-1H-benzotriazole-5-carboxylic acid

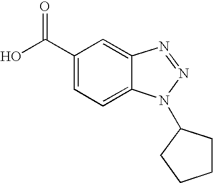

1-Cyclopentyl-1H-benzotriazole-5-carboxylic acid was prepared in a similar as described in Example 6.1 using 4-cyclopentylamino-3-nitro-benzoic acid as the intermediate. m/z (ES⁺): 232 [M+H]⁺. ¹H NMR (CD₃OD): 8.67 (dd, 1H, J₁=1.3, J₂=0.7, C(4)-H, 8.19 (dd, 1H, J₁=8.8, J₂=1.3, C(6)-H), 7.87 (dd, 1H, J₁=8.8, J₂=0.7, C(7)-H), 5.45-5.30 (m, 1H, NCH, 2.45-2.20 (m, 4H), 2.10-1.95 (m, 2H), 1.95-1.80 (m, 2H).

The intermediate 4-cyclopentylamino-3-nitro-benzoic acid was prepared in a manner as described in Example 6.1 a. using cyclopentylamine. ¹H NMR (CD₃OD): 8.81 (d, 1H, J=2.1, C(2)-H), 8.06 (dd, 1H, J₁=9.1, J₂=2.1, C(6)-H, 7.12 (d, 1H, J=9.1, C(5)-H), 4.2-4.1 (m, 1H, NHCH), 2.3-2.1 (m, 2H), 1.9-1.6 (m, 6H).

### Example 6.3

### Preparation of 1-(2′-Butyl)-1H-benzotriazole-5-carboxylic acid

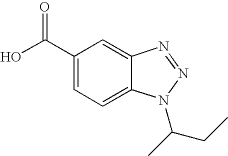

1-(2′-Butyl)-1H-benzotriazole-5-carboxylic acid was prepared in a similar as described in Example 6.1 using 4-(2′-butyl)amino-3-nitro-benzoic acid. m/z (ES⁺): 220 [M+H]⁺. ¹H NMR (CD₃OD): 8.72 (dd, 1H, J₁=1.3, J₂=0.7, C(4)-H), 8.22 (dd, 1H, J=8.8, J₂=1.3, C(6)-H), 7.91 (dd, 1H, J₁=8.8, J₂=0.7, C(7)-H, 5.10-5.00 (m, 1H, NCH), 2.30-2.05 (m, 2H, CH₂CH₃), 1.75 (d, 3H, J=6.8, CHCH₃), 0.85 (t, 3H, J=7.4, CH₂CH₃).

The intermediate 4-(2′-butyl)amino-3-nitro-benzoic acid was prepared in a similar manner as described in Example 6.1 a. using 2-butylamine. ¹H NMR (CD₃OD): 8.81 (d, 1H, J=2.1, C(2)-H, 8.04 (dd, 1H, J₁=9.2, J₂=2.1, C(6)-H), 7.10 (d, 1H, J=9.2, C(5)-H), 3.82 (sextet like, 1H, J=6.4, NHCH), 1.75-1.65 (m, 2H, CH₂CH₃), 1.31 (d, 3H, J=6.4, CHCH₃), 1.02 (t, 3H, J=7.5, CH₂CH₃).

### Example 6.4

### Preparation of 1-(3′-Pentyl)-1H-benzotriazole-5-carboxylic acid

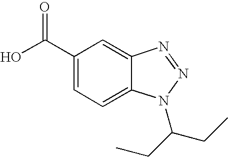

1-(3′-Pentyl)-1H-benzotriazole-5-carboxylic acid was prepared in a similar manner as described in Example 6.1 using 4-(3′-Pentyl)amino-3-nitro-benzoic acid. m/z (ES+): 234 [M+H]+. ¹H NMR (CD₃OD): 8.51 (dd, 1H, J1=1.4, J2=0.6, C(4)-H), 8.00 (dd, 1H, J1=8.8, J2=1.4, C(6)-H), 7.69 (dd, 1H, J1=8.8, J2=0.6, C(7)-H), 4.60 (septet like, 1H, 3=4.8, NCH), 2.10-1.85 (m, 4H, CH2CH3), 0.58 (t, 6H, J=7.4, CH2CH3).

The intermediate 4-(3′-pentyl)amino-3-nitro-benzoic acid was prepared in a similar manner as described in Example 6.1 a. using 3-pentylamine. ¹H NMR (CDCl3): 8.90 (d, 1H, J=2.1, C(2)-H), 8.35 (d, 1H, J=8.2, NH), 7.98 (dd, 1H, J1=9.2, J2=1.8, C(6)-H), 6.83 (d, 1H, J=9.2, C(5)-H), 3.50 (sextet like, 1H, J=7.6, NHCH), 1.75-1.50 (m, 4H, CH2CH3), 0.92 (t, 6H, J=7.4, CH2CH3).

### Example 6.5

### Preparation of 1-Cyclohexyl-1H-benzotriazole-5-carboxylic acid

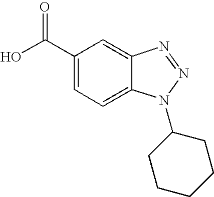

1-Cyclohexyl-1H-benzotriazole-5-carboxylic acid was prepared in a similar manner as described in Example 6.1 using 4-cyclohexylamino-3-nitro-benzoic acid. m/z (ES+): 246 [M+H]+. ¹H NMR (CD₃OD): 8.67 (s, 1H, C(4)-H), 8.19 (dd, 1H, J1=8.8, J2=1.4, C(6)-H), 7.90 (d, 1H, J=8.8, C(7)-H), 4.95-4.80 (m, 1H, NCH), 2.25-2.05 (m, 4H), 2.05-1.95 (m, 2H), 1.90-1.80 (m, 1H), 1.70-1.55 (m, 2H), 1.50-1.40 (m, 1H).

The intermediate 4-Cyclohexylamino-3-nitro-benzoic acid was prepared in a similar manner as described in Example 6.1 a. using cyclohexylamine. ¹H NMR (CD₃OD): 8.80 (d, 1H, J=2.1, C(2)-H), 8.03 (dd, 1H, J1=9.2, J2=2.1, C(6)-H), 7.10 (d, 1H, J=9.2, C(S)-H), 3.75-3.65 (m, 1H, NHCH), 2.10-2.05 (m, 2H), 1.85-1.75 (m, 2H), 1.75-1.60 (m, 1H), 1.60-1.30 (m, 5H).

### Example 6.6

### Preparation of 1-Benzyl-1H-benzotriazole-5-carboxylic acid

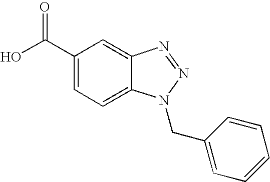 1-Benzyl-1H-benzotriazole-5-carboxylic acid was prepared in a similar manner as described in Example 6.1 using 4-benzylamino-3-nitro-benzoic acid. m/z (ES+): 254 [M+H]+. ¹H NMR (CD₃OD): 8.59 (dd, 1H, J1=1.3, J2=0.7, C(4)-H), 8.04 (dd, 1H, J1=8.8, J2=1.3, C(6)-H), 7.62 (dd, 1H, J1=8.8, J2=0.7, C(7)-H), 5.87 (d, 2H, NCH2).

The intermediate 4-benzylamino-3-nitro-benzoic acid was prepared in a similar manner as described in Example 6.1 a. using benzylamine. ¹H NMR (CDCl3): 9.00 (d, 1H, J=2.0, C(2)-H), 8.79 (t, 1H, J=5.5, NH), 8.06 (dd, 1H, J1=9.1, J2=1.8, C(6)-H), 7.5-7.3 (m, 5H), 6.89 (d, 1H, J=9.1, C(5)-H), 4.63 (d, 2H, NHCH2).

### Example 6.7

### Preparation of 1-Propyl-1H-benzotriazole-5-carboxylic acid

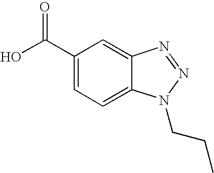

1-Propyl-1H-benzotriazole-5-carboxylic acid was prepared in a similar manner as described in Example 6.1 using 4-propylamino-3-nitro-benzoic acid. m/z (ES+): 206 [M+H]+. ¹H NMR (CD₃OD): 8.69 (dd, 1H, J1=1.4, J2=0.7, C(4)-H), 8.20 (dd, 1H, J1=8.8, J2=1.4, C(6)-H), 7.86 (dd, 1H, J1=8.8, J2=0.7, C(7)-H), 4.73 (t, 2H, J=7.0, NCH2), 2.06 (sextet like, 2H, J=7.2, CH2CH3), 0.96 (t, 3H, J=7.4, CH2CH3).

The intermediate 4-propylamino-3-nitro-benzoic acid was prepared in a similar manner as described in Example 6.1 a. using 1-propylamine. ¹H NMR (CD₃OD): 8.80 (d, 1H, J=2.1, C(2)-H), 8.04 (dd, 1H, J1=9.1, J2=2.1, C(6)-H), 7.07 (d, 1H, J=9.1, C(5)-H), 3.40 (t, 2H, J=7.1, NHCH2), 1.76 (sextet like, 2H, J=7.3, CH2CH3), 1.06 (t, 3H, J=7.4, CH2CH3).

### Example 6.8

### Preparation of 1-Cyclopropyl-1H-benzotriazole-5-carboxylic acid

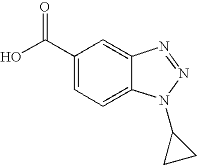

1-Cyclopropyl-1H-benzotriazole-5-carboxylic acid was prepared in a similar manner as described in Example 6.1 using 4-cyclopropylamino-3-nitro-benzoic acid. m/z (ES+): 204 [M+H]+. ¹H NMR (CD₃OD): 8.67 (dd, 1H, 31=1.4, J2=0.7, C(4)-H), 8.23 (dd, 1H, J1=8.7, J2=1.4, C(6)-H), 7.92 (dd, 1H, J1=8.7, J2=0.7, C(7)-H), 4.05-3.95 (m, 1H, NCH), 1.4-1.3 (m, 4H).

The intermediate 4-cyclopropylamino-3-nitro-benzoic acid was prepared in a similar manner as described in Example 6.1 a. using cyclopropylamine. ¹H NMR (CD₃OD): 8.78 (d, 1H, J=2.0, C(2)-H), 8.09 (dd, 1H, J1=9.0, J2=2.0, C(6)-H), 7.47 (d, 1H, J=9.0, C(5)-H), 2.71 (septet like, 1H, J=3.5, NHCH), 1.05-0.95 (m, 2H), 0.75-0.65 (m, 21).

### Example 6.9

### Preparation of 1-(3′-Isopropoxy-propyl)-1H-benzotriazole-5-carboxylic acid

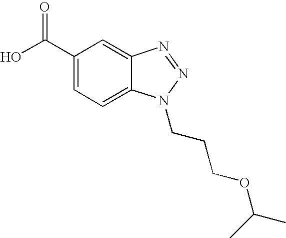

1-(3′-Isopropoxy-propyl)-1H-benzotriazole-5-carboxylic acid was prepared in a similar manner as described in Example 6.1 using 4-(3′-isopropoxy-propyl)amino-3-nitro-benzoic acid. m/z (ES+): 264 [M+H]+. ¹H NMR (CD₃OD): 8.68 (dd, 1H, J1=1.4, J2=0.7, C(4)-H), 8.20 (dd, 1H, J1=8.8, J2=1.4, C(6)-H), 7.86 (dd, 1H, J1=8.8, J2=0.7, C(7)-H), 4.85 (t, 2H, NCH2), 3.49 (septet, 1H, J=6.1, CH(CH3)2), 3.41 (t, 2H, J=5.8, CH2O), 2.25 (quintet like, 2H, J=5.9, CH2CH2CH2), 1.07 (d, 6H, J=6.1, CH(CH3)2).

The intermediate 4-(3′-isopropoxy-propyl)amino-3-nitro-benzoic acid was prepared in a similar manner as described in Example 6.1 a. using 3-isopropoxypropyl amine. ¹H NMR (CD₃OD): 8.80 (d, 1H, J=2.1, C(2)-H), 8.04 (dd, 1H, J1=9.1, J2=2.1, C(6)-H), 7.09 (d, 1H, J=9.1, C(5)-H), 3.65-3.55 (m, 3H, NHCH2 & CH(CH3)2), 3.53 (t, 2H, J=6.5, CH2O), 1.97 (quintet like, 2H, J=6.1, CH2CH2CH2), 1.18 (d, 6H, J=6.1, CH(CH3)2).

### Example 6.10

### Preparation of 1-(Tetrahydro-furan-2′-ylmethyl)-1H-benzotriazole-5-carboxylic acid.

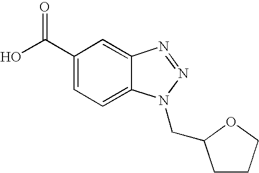

1-(Tetrahydro-furan-2′-ylmethyl)-1H-benzotriazole-5-carboxylic acid was prepared in a similar manner as described in Example 6.1 using 4-(tetrahydro-furan-2′-ylmethyl)amino-3-nitro-benzoic acid. m/z (ES+): 248 [M+H]+. ¹H NMR (CD₃OD): 8.67 (s, 1H, C(4)-H), 8.18 (dd, 1H, J1=8.7, J2=1.4, C(6)-H), 7.90 (dd, 1H, J1=8.8, J2=0.4, C(7)-H), 4.95-4.85 (m, 1H), 4.79 (dd, 1H, J1=14.6, J2=6.5), 4.42 (ddd, 1H, J1=13.4, J2=6.6, J3=3.6), 3.80-3.60 (m, 2H), 2.20-2.05 (m, 1H0, 1.95-1.55 (m, 3H).

The intermediate 4-(tetrahydro-furan-2′-ylmethyl)amino-3-nitro-benzoic acid was prepared in a similar manner as described in Example 6.1 a. using tetrahydro-furan-2-ylmethyl)amine. ¹H NMR (CD₃OD): 8.78 (d, 1H, J=2.1, C(2)-H), 8.03 (dd, 1H, J1=9.1, J2=2.1, C(6)-H), 7.11 (d, 1H, J=9.1, C(5)-H), 4.25-4.15 (m, 1H), 3.91 (dd, 1H, J1=15.0, J2=6.7), 3.79 (dd, 1H, J1=13.9, J2=7.0), 3.58 (dd, 1H, J1=13.5, J2=3.8), 3.42 (dd, 1H, J1=13.5, J2=7.0), 2.15-2.05 (m, 1H0, 2.05-1.85 (m, 2H), 1.80-1.70 (m, 1H).

### Example 6.11

### Preparation of 1-Cyclobutyl-1H-benzotriazole-5-carboxylic acid

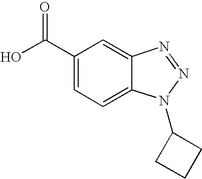

1-Cyclobutyl-1H-benzotriazole-5-carboxylic acid was prepared in a similar manner as described in Example 6.1 using 4-cyclobutylamino-3-nitro-benzoic acid. m/z (ES+): 218 [M+H]+. ¹H NMR (CD₃OD): 8.68 (dd, 1H, J1=1.4, J2=0.6, C(4)-H), 8.19 (dd, 1H, J1=8.8, J2=1.4, C(6)-H), 7.86 (dd, 1H, J1=8.8, J2=0.6, C(7)-H), 5.46 (quintet like, 1H, J=8.3, NCH), 2.95-2.80 (m, 2H), 2.80-2.65 (m, 2H), 2.15-2.05 (m, 2H).

The intermediate 4-cyclobutylamino-3-nitro-benzoic acid was prepared in a similar manner as described in Example 6.1 a. using cyclobutylamine. ¹H NMR (CD₃OD): 8.78 (d, 1H, J=2.0, C(2)-H), 8.03 (dd, 1H, J1=9.0, J2=2.0, C(6)-H), 6.93 (d, 1H, J=9.0, C(5)-H), 4.22 (quintet like, 1H, J=7.8, NHCH), 2.60-2.50 (m, 2H), 2.15-2.00 (m, 2H), 2.00-1.85 (m, 2H).

### Example 6.12

### Preparation of 1-(2-Methoxy-ethyl)-1H-benzotriazole-5-carboxylic acid

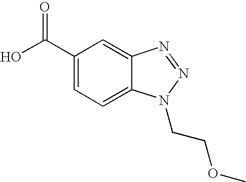 1-(2-Methoxy-ethyl)-1H-benzotriazole-5-carboxylic acid was prepared in a similar manner as described in Example 6.1 using 4-(2′-methoxy-ethyl)amino-3-nitro-benzoic acid. m/z (ES+): 222 [M+H]+. ¹H NMR (CD₃OD): 8.67 (dd, 1H, J1=1.4, J2=0.7, C(4)-H), 8.18 (dd, 1H, J1=8.8, J2=1.4, C(6)-H), 7.87(dd, 1H, 1=8.8, J2=0.7, C(7)-H), 4.93 (t, 2H, J=5.1, NCH2), 3.91 (t, 3H, J=5.1, OCH2), 3.29 (s, 3H, OCH3).

The intermediate 4-(2′-methoxy-ethyl)amino-3-nitro-benzoic acid was prepared in a similar manner as described in Example 6.1 a. using 2-methoxyethylamine. ¹H NMR (CD₃OD): 8.80 (d, 1H, J=2.1, C(2)-H), 8.05 (dd, 1H, J1=9.1, J2=2.1, C(6)-H), 7.10 (d, 1H, J=9.1, C(5)-H), 3.69 (t, 2H, J=5.2, NHCH2), 3.60 (t, 3H, J=5.2, OCH2), 3.42 (s, 3H, OCH3).

### Example 6.13

### Preparation of 1-(3′Methoxybenzyl)-1H-benzotriazole-5-carboxylic acid

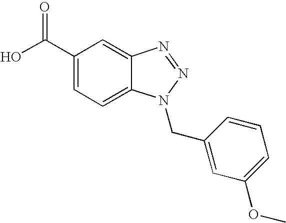

1-(3′Methoxybenzyl)-1H-benzotriazole-5-carboxylic acid was prepared in a similar manner as described in Example 6.1 using 4-(3′methoxybenzyl)amino-3-nitro-benzoic acid. m/z (ES+): 284 M+H]+. ¹H NMR (CD₃OD): 8.69 (dd, 1H, J1=1.4, J2=0.7, C(4)-H), 8.15 (dd, 1H, J1=8.7, J2=1.4, C(6)-H), 7.72 (dd, 1H, J1=8.7, J2=0.7, C(7)-H), 7.26 (t, 1H, J=7.9, C(5′)-H), 7.0-6.8 (m, 3H), 5.94 (s, 2H, NCH2), 3.75 (s, 3H, OCH3).

The intermediate 4-(3′methoxybenzyl)amino-3-nitro-benzoic acid was prepared in a similar manner as described in Example 6.1 a. using 3-methoxybenzylamine. ¹H NMR (CD₃OD): 8.82 (d, 1H, J=2.1, C(2)-H), 7.97 (dd, 1H, J1=9.1, J2=2.1, C(6)-H), 7.27 (t, 1H, J=8.1, C(5′)-H), 7.0-6.9 (m, 3H), 6.84 (dd, 1H, J1=7.5, J2=2.5, C(5)-H), 4.64 (s, 2H, NHCH12), 3.78 (s, 3H, OCH3).

### Example 6.14

### Preparation of 1-(4′Methoxybenzyl)-1H-benzotriazole-5-carboxylic acid

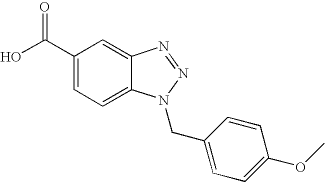

1-(4′Methoxybenzyl)-1H-benzotriazole-5-carboxylic acid was prepared in a similar manner as described in Example 6.1 using 4-(4′methoxybenzyl)amino-3-nitro-benzoic acid. m/z (ES+): 284 [M+H]+. ¹H NMR (CD₃OD): 8.68 (s, 1H, C(4)-H), 8.14 (dd, 1H, J1=8.8, J2=1.4, C(6)-H), 7.72 (d, 1H, J=8.8, C(7)-H), 7.31 (d, 2H, J=8.7, C(2′)-H), 6.90 (d, 2H, J=8.7, C(2′)-H), 5.90 (s, 2H, NCH2), 3.76 (s, 3H, OCH3).

The intermediate 4-(4′methoxybenzyl)amino-3-nitro-benzoic acid was prepared in a similar manner as described in Example 6.1 a. using 4-methoxybenzylamine. ¹H NMR (CD₃OD): 8.81 (d, 1H, J=2.0, C(2)-H), 7.98 (dd, 1H, J1=9.0, J2=2.0, C(6)-H), 7.31 (d, 2H, J=8.8, C(2′)-H), 7.00 (d, 1H, 1=9.0, C(5)-H), 6.91 (d, 2H, J=8.8, C(3′)-H) 4.59 (s, 2H, NHCH2), 3.78 (s, 3H, OCH3).

### Example 6.15

### Preparation of 1-[2′-(4″-Methoxy-phenyl)-ethylamino]-1H-benzotriazole-5-carboxylic acid

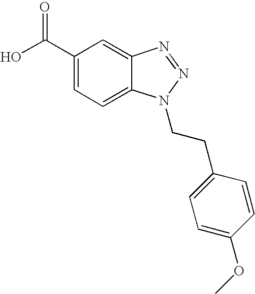

1-[2′-(4″-Methoxy-phenyl)-ethylamino]-1H-benzotriazole-5-carboxylic acid was prepared in a similar manner as described in Example 6.1 using 4-[2′-(4″-methoxy-phenyl)-ethylamino]-3-nitro-benzoic acid. m/z (ES+): 298 [M+H]+. ¹H NMR (CD₃OD): 8.41 (s, 1H, C(4)-H), 7.84 (dd, 1H, J1=8.8, J2=1.3, C(6)-H), 7.29 (d, 1H, J=8.8, C(7)-H), 6.72 (d, 2H, J=8.6, C(2″)-H), 6.50 (d, 2H, J=8.6, C(3″)-H), 4.73 (t, 2H, J=6.8, NCH2), 3.48 (s, 3H, OCH3), 5.90 (t, 2H, J=6.8, NCH2CH2).

The intermediate 4-[2′-(4″-methoxy-phenyl)-ethylamino]-3-nitro-benzoic acid was prepared in a similar manner as described in Example 6.1 a. using 2-(4-methoxy-phenyl)-ethylamine. ¹H NMR (CD₃OD): 8.77 (d, 1H, J=2.0, C(2)-H), 8.02 (dd, 1H, J1=9.1, J2=2.0, C(6)-H), 7.20 (d, 2H, J=8.6, C(2″)-H), 7.06 (d, 1H, J=9.1, C(5)-H), 6.87 (d, 2H, J=8.6, C(3′)-H) 3.78 (s, 3H, OCH3), 3.65 (t, 2H, J=7.0, NHCH2), 2.96 (t, 2H, J=7.0, NHCH2CH2).

### Example 6.16

### Preparation of 1-[2′-(3″-Methoxy-phenyl)-ethylamino]-1H-benzotriazole-5-carboxylic acid

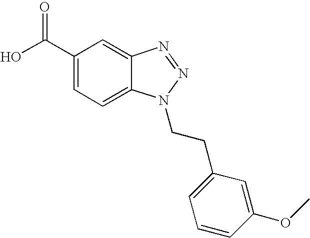

1-[2′-(3″-Methoxy-phenyl)-ethylamino]-1H-benzotriazole-5-carboxylic acid was prepared in a similar manner as described in Example 6.1 using 4-[2′-(4″-methoxy-phenyl)-ethylamino]-3-nitro-benzoic acid. m/z (ES+): 298 [M+H]+. ¹H NMR (CD₃OD): 8.61 (dd, 1H, J1=1.4, J2=0.7, C(4)-H), 8.03 (dd, 1H, J1=8.8, J2=1.4, C(6)-H), 7.48 (dd, 1H, J1=8.8, J2=0.7, C(7)-H), 7.06 (t, 1H, J=7.9, C(5″)-H), 6.69 (ddd, 1H, J1=8.3, J2=2.5, J2=0.6, C(6″)-H), 6.61 (d, 1H, J=7.5, C(4″)-H), 6.53 (t, 1H, J=2.0, C(2″)-H), 4.98 (t, 2H, J=6.8, NCH2), 3.62 (s, 3H, OCH3), 3.27 (t, 2H, J=6.8, NCH2CH2).

The intermediate 4-[2′-(3″-methoxy-phenyl)-ethylamino]-3-nitro-benzoic acid was prepared in a similar manner as described in Example 6.1 a. using 2-(3-methoxy-phenyl)-ethylamine. ¹H NMR (CD₃OD): 8.77 (d, 1H, J=2.1, C(2)-H), 8.02 (dd, 1H, J1=9.1, J2=2.1, C(6)-H), 7.22 (t, 1H, J=8.6, C(5″)-H), 7.06 (d, 1H, J=9.1, C(S)-H), 6.90-6.85 (m, 2H) 6.85-6.75 (m, 1H), 3.78 (s, 3H, OCH3), 3.68 (t, 2H, J=7.0, NHCH2), 3.00 (t, 2H, J=7.0, NHCH2CH2).

### Example 6.17

### Preparation of 1-(3′,5′-Difluorobenzyl)-1H-benzotriazole-5-carboxylic acid

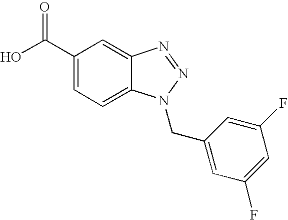

1-(3′,5′-Difluorobenzyl)-1H-benzotriazole-5-carboxylic acid was prepared in a similar manner as described in Example 6.1 using 4-(3′,5′-difluorobenzyl)amino-3-nitro-benzoic acid. m/Z (ES+): 290 M+H]+. ¹H NM (CD₃OD): 8.72 (dd, 1H, J1=1.4, J2=0.6, C(4)-H), 8.20 (dd, 1H, J1=8.8, J2=1.4, C(6)-H), 7.78 (dd, 1H, J1=8.8, J2=0.6, C(7)-H), 7.00-6.90 (m, 3H), 6.00 (s, 2H, NCH2).

The intermediate 4-(3′,5′-difluorobenzyl)amino-3-nitro-benzoic acid was prepared in a similar manner as described in Example 6.1 a. using 3,5-difluoro-benzylamine. ¹H NMR (CD₃OD): 8.34 (d, 1H, J=2.0, C(2)-H), 7.99 (dd, 1H, J1=8.6, J2=2.0, C(6)-H), 7.05-6.85 (m, 4H), 4.71 (s, 2H, NHCH2).

### Example 6.18

### Preparation of 1-(2-Ethylsulfanyl-ethyl)-1H-benzotriazole-5-carboxylic acid

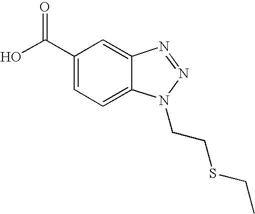

1-(2-Ethylsulfanyl-ethyl)-1H-benzotriazole-5-carboxylic acid was prepared in a similar manner as described in Example 6.1 using 4-(2-ethylsulfanyl-ethylamino)-3-nitro-benzoic acid. ¹H NMR (CD₃OD): 8.69 (d, 1H, J=0.8, C(4)-H), 8.21 (dd, 1H, J1=8.8, J2=1.4, C(6)-H), 7.89 (d, 1H, J=8.8, C(7)-H), 4.95 (t, 2H, J=6.8, NCH2), 3.18 (t, 2H, J=6.8, NCH2CH2), 2.48 (q, 2H, J=7.6, CH2CH3), 1.18 (t, 3H, J=7.4, CH2CH3).

The intermediate 4-(2-ethylsulfanyl-ethylamino)-3-nitro-benzoic acid was prepared in a similar manner as described in Example 6.1 a. using 2-ethylsulfanyl-ethylamine. ¹H NMR (CDCl₃): 8.99 (d, 1H, J=1.9, C(2)-H, 8.68 (br s, 1H, NH, 8.12 (dd, 1H, J₁=9.0, J₂=1.9, C(6)-H, 6.97 (d, 1H, J=9.1, C(5)-H), 3.61 (q like, 2H, J=6.3, NHCH₂), 2.91 (t, 2H, J=6.8, NCH₂CH2), 2.63 (q, 2H, J=7.4, CH₂CH₃), 1.32 (t, 3H, J=7.4, CH₃).

### Example 6.19

### Preparation of 1-t-Butyl-1H-benzotriazole-5-carboxylic acid

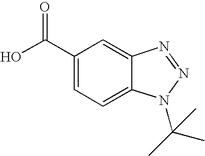

1-t-Butyl-1H-benzotriazole-5-carboxylic acid was prepared in a similar manner as described in Example 6.1 using 4-tert-butylamino-3-nitro-benzoic acid. ¹H NMR (CD₃OD): 8.68 (dd, 1H, J1=1.5, J2=0.6, C(4)-H), 8.17 (dd, 1H, J1=8.9, J2=1.5, C(6)-H), 8.07 (dd, 1H, J1=8.8, J2=0.6, C(7)-H), 1.90 (s, 9H, CH3).

The intermediate 4-tert-butylamino-3-nitro-benzoic acid was prepared in a similar manner as described in Example 6.1 a. using tert-butylamine. ¹H NMR (CD₃OD): 9.04 (d, 1H, J=2.1, C(2)-H, 8.25 (dd, 1H, J₁=9.2, J₂=2.1, C(6)-H, 7.54 (d, 1H, J=9.2, C(5)-H), 1.78 (s, 9H, CH₃).

### Example 6.20

### Preparation of 1-(3′-Hydroxy-propyl)-1H-benzotriazole-5-carboxylic acid

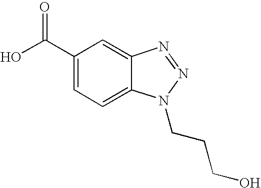

1-(3′-Hydroxy-propyl)-1H-benzotriazole-5-carboxylic acid was prepared in a similar manner as described in Example 6.1 using 4-(3-hydroxy-propylamino)-3-nitro-benzoic acid. ¹H NMR (CD₃OD): 8.69 (dd, 1H, J1=1.4, J2=0.7, C(4)-H), 8.21 (dd, 1H, J1=8.8, J2=1.4, C(6)-H), 7.88 (dd, 1H, J1=8.8, J2=0.7, C(7)-H), 4.87 (t, 2H, J=6.8, NCH2), 3.59 (t, 2H, J=6.0, CH2OH), 2.22 (quintet like, 2H, J=6.5, CH2CH2CH2).

The intermediate 4-(3-hydroxy-propylamino)-3-nitro-benzoic acid was prepared in a similar manner as described in Example 6.1 a. using 3-hydroxy-propylamine. ¹H NMR (CD₃OD): 8.81 (d, 1H, J=1.9, C(2)-H), 8.05 (dd, 1H, J₁=9.1, J₂=1.9, C(6)-H, 7.11 (d, 1H, J=9.1, C(5)-H), 3.73 (t, 2H, J=5.9, CH₂OH), 3.55 (t, 2H, J=6.8, NHCH₂), 1.94 (quintet like, 2H, J=6.3, CH₂CH₂OH).

### Example 6.21

### Preparation of 1-(1′,3′-Dimethyl-butyl)-1H-benzotriazole-5-carboxylic acid

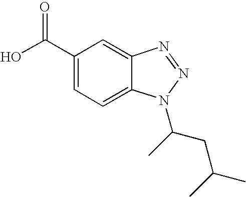

1-(1′,3′-Dimethyl-butyl)-1H-benzotriazole-5-carboxylic acid was prepared in a similar manner as described in Example 6.1 using 1-(1′,3′-dimethyl-butyl)-1H-benzotriazole-5-carboxylic acid. ¹H NMR (CD₃OD): 8.69 (d, 1H, J=0.8, C(4)-H), 8.20 (dd, 1H, J1=8.8, J2=1.4, C(6)-H), 7.91 (d, 1H, J=8.8, C(7)-H), 5.25-5.15 (m, 1H, NCH), 2.35-2.20 (m, 1H), 1.90-1.75 (m, 1H), 1.70 (d, 3H, J=6.4, NCHCH3), 1.30-1.15 (m, 1H), 0.95 (d, 3H, J=6.8, CH3), 0.85 (d, 3H, J=6.4, CH3).

The intermediate 1-(1′,3′-dimethyl-butyl)-1H-benzotriazole-5-carboxylic acid was prepared in a similar manner as described in Example 6.1 a. using 1,3-dimethyl-butylamine. ¹H NMR (CD₃OD): 8.69 (d, 1H, J=0.8, C(4)-H, 8.20 (dd, 1H, J₁=8.8, J₂=1.4, C(6)-H, 7.91 (d, 1H, J=8.8, C(7)-H, 5.25-5.15 (m, 1H, NCH, 2.35-2.20 (m, 1H), 1.90-1.75 (m, 1H), 1.70 (d, 3H, J=6.4, NCHCH₃), 1.30-1.15 (m, 1H), 0.95 (d, 3H, J=6.8, CH₃), 0.85 (d, 3H, J=6.4, CH₃).

### Example 6.22

### Preparation of 1-(3′,3′-Dimethyl-butyl)-1H-benzotriazole-5-carboxylic acid

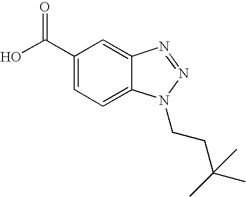

1-(3′,3′-Dimethyl-butyl)-1H-benzotriazole-5-carboxylic acid was prepared in a similar manner as described in Example 6.1 using 1-(3′,3′-dimethyl-butyl)-1H-benzotriazole-5-carboxylic acid. ¹H NMR (CD₃OD): 8.68 (s, 1H, C(4)-H), 8.22 (dd, 1H, J1=8.8, J2=1.4, C(6)-H), 7.84 (dd, 1H, J1=8.8, J2=0.6, C(7)-H), 4.85-4.75 (m, 2H, NCH2), 1.96-1.90 (m, 2H, NCH2CH2), 1.05 (s, 6H, CH3).

The intermediate 1-(3′,3′-dimethyl-butyl)-1H-benzotriazole-5-carboxylic acid was prepared in a similar manner as described in Example 6.1 a. using 3,3-dimethyl-butylamine. ¹H NMR (CD₃OD): 8.68 (s, 1H, C(4)-H, 8.22 (dd, 1H, J₁=8.8, J₂=1.4, C(6)-H), 7.84 (dd, 1H, J₁=8.8, J₂=0.6, C(7)-H), 4.85-4.75 (m, 2H, NCH₂), 1.96-1.90 (m, 2H, NCH₂CH₂), 1.05 (s, 6H, CH₃).

### Example 6.23

### Preparation of 1-Heptyl-1H-benzotriazole-5-carboxylic acid

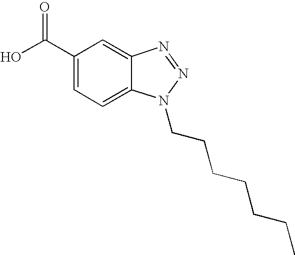

1-Heptyl-1H-benzotriazole-5-carboxylic acid was prepared in a similar manner as described in Example 6.1 using 4-heptylamino-3-nitro-benzoic acid. ¹H NMR (CD₃OD): 8.67 (s, 1H, C(4)-H), 8.20 (dd, 1H, J1=8.7, J2=1.3, C(6)-H), 7.83 (d, 1H, J=8.7, C(7)-H), 4.75 (t, 2H, J=6.8, NCH2), 2.05-2.00 (m, 2H, NCH2CH2), 1.5-1.3 (m, 8H), 1.0-0.8 (m, 3H).

The intermediate 4-heptylamino-3-nitro-benzoic acid was prepared in a similar manner as described in Example 6.1 a. using heptylamine. ¹H NMR (CDCl₃): 8.94 (d, 1H, J=2.0, C(2)-H), 8.42 (t, 1H, J=4.9, NH), 8.07 (dd, 1H, J₁=9.1, J₂=2.0, C(6)-H), 6.88 (d, 1H, J=9.1, C(5)-H), 3.36 (q like, 2H, J=6.5, NHCH₂), 1.76 (quintet like, 2H, J=7.3, NCH₂CH₂), 1.5-1.3 (m, 8H), 1.0-0.8 (m, 3H).

### Example 6.24

### Preparation of 1-(2′-Methoxy-1′-methyl-ethyl)-1H-benzotriazole-5-carboxylic acid

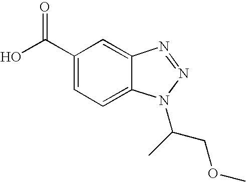

1-(2‘-Methoxy-1 ’-methyl-ethyl)-1H-benzotriazole-5-carboxylic acid was prepared in a similar manner as described in Example 6.1 using 4-(2′-methoxy-1′-methyl-ethyl)amino-3-nitro-benzoic acid. ¹H NMR (CD₃OD): 8.68 (s, 1H, C(4)-H), 8.18 (dd, 1H, J1=8.8, J2=1.3, C(6)-H), 7.88 (d, 1H, J=8.8, C(7)-H), 5.35-5.25 (m, 1H, NCH), 3.93 (dd, 1H, J1=10.0, J2=8.4, CHCHH), 3.85 (dd, 1H, J1=10.0, J2=4.4, CHCHH), 3.25 (s, 3H, OCH3), 1.73 (d, 3H, J=6.8, CH3).

The intermediate 4-(2′-methoxy-1′-methyl-ethyl)amino-3-nitro-benzoic acid was prepared in a similar manner as described in Example 6.1 a. using 2-methoxy-1-methyl-ethylamine. ¹H NMR (CD₃OD): 8.80 (d, 1H, J=2.1, C(2)-H), 8.04 (dd, 1H, J₁=9.1, J₂=2.1, C(6)-H), 7.13 (d, 1H, J=9.1, C(5)-H), 4.08 (sextet like, 1H, J=5.4, NHCH), 3.60-3.50 (m, 2H, CHCH₂), 3.41 (s, 3H, OCH₃), 1.33 (d, 3H, J=6.8, CH₃).

### Example 6.25

### Preparation of 1-(2′-Hydroxy-1′-hydroxymethyl-ethyl)-1H-benzotriazole-5-carboxylic acid

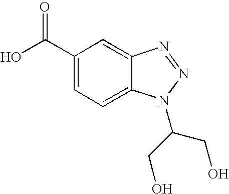

1-(2′-Hydroxy-1′-hydroxymethyl-ethyl)-1H-benzotriazole-5-carboxylic acid was prepared in a similar manner as described in Example 6.1 using 4-(2′-hydroxy-1′-hydroxymethyl-ethyl)amino-3-nitro-benzoic acid. ¹H NMR (CD₃OD): 8.68 (s, 1H, C(4)-H), 8.19 (dd, 1H, J1=8.8, J2=1.4, C(6)-H), 7.88 (d, 1H, J=8.8, C(7)-H), 5.10-5.00 (m, 1H, NCH), 4.25-4.10 (m, 4H, CH2OH).

The intermediate 4-(2′-hydroxy-1′-hydroxymethyl-ethyl)amino-3-nitro-benzoic acid was prepared in a similar manner as described in Example 6.1 a. using 2-hydroxy-1-hydroxymethyl-ethylamine. m/z (ES⁺): 257 [M+H]⁺. ¹H NMR (CD₃OD): 8.81 (d, 1H, J=2.1, C(2)-H), 8.03 (dd, 1H, J₁=9.2, J₂=2.1, C(6)-H), 7.18 (d, 1H, J=9.2, C(5)-H), 3.90 (quintet like, 1H, J=5.0, NHCH), 3.85-3.70 (r, 4H, CH₂OH).

### Example 6.26

### Preparation of 1-Ethyl-1H-benzotriazole-5-carboxylic acid

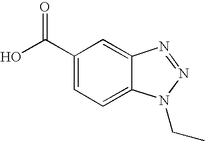

1-Ethyl-1H-benzotriazole-5-carboxylic acid was prepared in a similar manner as described in Example 6.1 using 4-ethylamino-3-nitro-benzoic acid. ¹HNMR (CD₃OD): 8.69 (d, 1H, J=0.7, C(4)-H), 8.21 (dd, 1H, J1=8.8, J2=1.3, C(6)-H), 7.86 (dd, 1H, J1=8.8, J2=0.7, C(7)-H), 4.80 (q, 2H, J=7.4, NCH2), 1.63 (t, 3H, J=7.4, CH3).

The intermediate 4-ethylamino-3-nitro-benzoic acid was prepared in a similar manner as described in Example 6.1 a. using 2-methoxy-1-methyl-ethylamine. ¹H NMR (CD₃OD): 8.80 (d, 1H, J=2.1, C(2)-H, 78.05 (dd, 1H, J₁=9.1, J₂=2.1, C(6)-H), 7.07 (d, 1H, J=9.1, C(5)-H, 3.48 (q, 2H, J=7.2, NHCH₂), 1.35 (t, 3H, J=7.2, CH₃).

### Example 6.27

### Preparation of 1-Pentyl-1H-benzotriazole-5-carboxylic acid

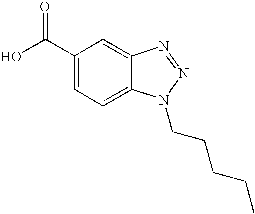

1-Pentyl-1H-benzotriazole-5-carboxylic acid was prepared in a similar manner as described in Example 6.1 using 4-pentylamino-3-nitro-benzoic acid. ¹H NMR (CD₃OD): 8.69 (d, 1H, J=0.8, C(4)-1H), 8.21 (dd, 1H, J1=8.8, J2=1.4, C(6)-H), 7.86 (dd, 1H, J1=8.8, J2=0.6, C(7)-H), 4.76 (q, 2H, J=7.2, NCH2), 2.10-1.95 (m, 2H, NCH2CH2), 1.45-1.25 (m, 4H), 0.90 (t, 3H, J=7.2, CH3).

The intermediate 4-pentylamino-3-nitro-benzoic acid was prepared in a similar manner as described in Example 6.1 a. using pentylamine. ¹H NMR (CD₃OD): 8.66 (d, 1H, J=2.1, C(2)-H), 7.91 (dd, 1H, J₁=9.1, J₂=2.1, C(6)-H), 6.93 (d, 1H, J=9.1, C(5)-H), 3.29 (t, 2H, J=7.2, NHCH₂), 1.62 (quintet like, 2H, J=7.0, NCH₂CH₂), 1.35-1.25 (m, 4H), 0.83 (t, 3H, J=7.1, CH₃).

### Example 6.28

### Preparation of 1-(2′,2′-Dimethyl-propyl)-1H-benzotriazole-5-carboxylic acid

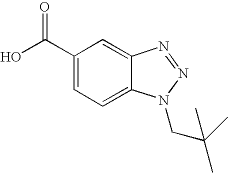

1-(2′,2′-Dimethyl-propyl)-1H-benzotriazole-5-carboxylic acid was prepared in a similar manner as described in Example 6.1 using 4-(2′,2′-dimethyl-propyl)amino-3-nitro-benzoic acid. ¹H NMR (CD₃OD): 8.69 (dd, 1H, J1=1.4, J2=0.7, C(4)-H), 8.20 (dd, 1H, J1=8.8, J2=1.4, C(6)-H), 7.86 (dd, 1H, J1=8.8, J2=0.7, C(7)-H), 4.56 (s, 2H, NCH2), 1.05 (s, 9H, CH3).

The intermediate 4-(2′,2′-dimethyl-propyl)amino-3-nitro-benzoic acid was prepared in a similar manner as described in Example 6.1 a. using 2,2-dimethyl-propylamine. ¹H NMR (CD₃OD): 8.81 (d, 1H, J=2.1, C(2)-H), 8.04 (dd, 1H, J₁=9.1, J₂=2.1, C(6)-H), 7.13 (d, 1H, J=9.1, C(5)-H), 3.25 (s, 2H, NHCH₂), 1.08 (s, 9H, CH₃).

### Example 6.29

### Preparation of 1-(2′-Ethoxy-ethyl)-benzotriazole-5-carboxylic acid

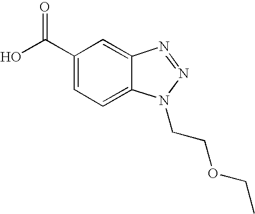

1-(2′-Ethoxy-ethyl)-1H-benzotriazole-5-carboxylic acid was prepared in a similar manner as described in Example 6.1 using 42′-ethoxy-ethyl)amino-3-nitro-benzoic acid. ¹H NMR (CD₃OD): 8.68 (d, 1H, J=0.8, C(4)-H), 8.18 (dd, 1H, J1=8.8, J2=1.3, C(6)-H), 7.88 (dd, 1H, J1=8.8, J2=0.6, C(7)-H), 4:92 (t, 2H, J=5.2, NCH2), 3.95 (t, 2H, J=5.0, NCH2CH2), 3.44 (q, 2H, J=7.0, CH2CH3), 1.04 (t, 3H, J=7.0, CH3).

The intermediate 4-(2′-ethoxy-ethyl)amino-3-nitro-benzoic acid was prepared in a similar manner as described in Example 6.1 a. using 2-ethoxy-ethylamine. ¹H NMR (CD₃OD): 8.80 (d, 1H, J=2.0, C(2)-H), 8.05 (dd, 1H, J₁=9.1, J₂=2.0, C(6)-H), 7.10 (d, 1H, J=9.1, C(5)-H), 3.74 (t, 2H J=7.0, NHCH₂CH₂), 3.65-3.55 (m, 4H, NHCH₂& CH₂CH₃), 1.22 (t, 3H, J=7.0, CH₃).

### Example 6.30

### Preparation of 1-(1′,2′-Dimethyl-propyl)-11H-benzotriazole-5-carboxylic acid

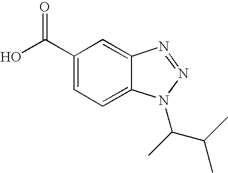

1-(1′,2′-Dimethyl-propyl)-1H-benzotriazole-5-carboxylic acid was prepared in a similar manner as described in Example 6.1 using 4-(1′,2′-dimethyl-propyl)amino-3-nitro-benzoic acid. ¹H NMR (CD₃OD): 8.69 (dd, 1H, J1=1.4, J2=0.5, C(4)-H), 8.19 (dd, 1H, J1=8.8, J2=1.4, C(6)-H), 7.88 (dd, 1H, J1=8.8, J2=0.5, C(7)-H), 4.85-4.75 (m, 1H, NCH), 2.45-2.35 (m, 1H, J=5.0, CH(CH3)2), 1.74 (d, 3H, J=6.4, NCHCH3), 1.09 (d, 3H, J=6.8, CH3), 0.75 (d, 3H, J=6.8, CH3).

The intermediate 4-(1′,2′-dimethyl-propyl)amino-3-nitro-benzoic acid was prepared in a similar manner as described in Example 6.1 a. using 1,2-dimethyl-propylamine. ¹H NMR (CD₃OD): 8.81 (d, 1H, J=2.1, C(2)-H), 8.04 (dd, 1H, J₁=9.1, J₂=2.1, C(6)-H), 7.11 (d, 1H, J=9.1, C(5)-H), 3.78 (quintet like, 1H, NHCH, 2.00-1.90 (m. 1H, CH(CH₃)₂), 1.27 (d, 3H, J=6.5, NCHCH₃), 1.06 (d, 3H, J=6.9, CH₃), 1.01 (d, 3H, J=6.8, CH₃).

### Example 6.31

### Preparation of 1-Benzhydryl-1H-benzotriazole-5-carboxylic acid

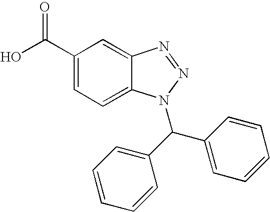

1-Benzhydryl-1H-benzotriazole-5-carboxylic acid was prepared in a similar manner as described in Example 6.1 using 4-benzhydrylamino-3-nitro-benzoic acid. ¹H NMR (CD₃OD): 8.72 (s, 1H, C(4)-H), 8.07 (d, 1H, J=8.8, C(6)-H), 7.48 (d, 1H, J=8.8, C(7)-H), 7.40-7.20 (m, 10H), 4.96 (s, 1H, NCH).

The intermediate 4-benzhydrylamino-3-nitro-benzoic acid was prepared in a similar manner as described in Example 6.1 a. using benzhydrylamine. ¹H NMR (CD₃OD): 8.69 (d, 1H, J=2.0, C(2)-H), 7.79 (dd, 1H, J₁=9.1, J₂=2.0, C(6)-H), 7.30-7.15 (m, 10H), 6.77 (d, 1H, J=9.1, C(5)-H), 5.88 (br s, 1H, NHCH).

### Example 6.32

### Preparation of 1-Allyl-1H-benzotriazole-5-carboxylic acid

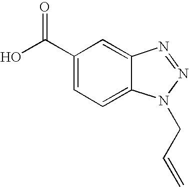

Benzotriazole-5-carboxylic acid (0.163 g, 1.0 mmol), allyl bromide (0.18 g, 1.5 mmol) and potassium carbonate (0.304 g, 2.2 mmol) were stirred for 18 hours at 60° C. in DMA (3 mL). The resulting solution was diluted with water and acetonitrile until all solid was dissolved, and purified by preparative HPLC to give 1-(allyl)-1H-benzotriazole-5-carboxylic acid m/z (ES+): 204 [+H]+. ¹H NMR (CD₃OD): 8.73 (s, 1H, C(4)-H), 8.19 (dd, 1H, J1=8.8, J2=1.6, C(6)-H), 7.85 (d, 1H, J=8.4, C(7)-H), 6.1-6.0 (m, 1H, CH═CH2), 5.50-5.40 (m, 1H, CH═CHH trans to H), 5.35-5.30 (m, 1H, CH═CHH cis to H), 4.90-4.85 (m, 2H, NCH2).

### Example 6.33

### Preparation of 1-Butyl-1H-benzotriazole-5-carboxylic acid

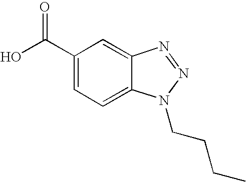

1-Butyl-1H-benzotriazole-5-carboxylic acid was prepared in a similar manner as described in Example 6.32 using butyl bromide. m/z (ES+): 220 M+H]+. ¹H NMR (CD₃OD): 8.68 (dd, 1H, J1=1.4, J2=0.6, C(4)-H), 8.20 (dd, 1H, J1=8.8, J2=1.4, C(6)-H), 7.86 (dd, 1H, J1=8.4, J2=0.6, C(7)-H), 4.76 (t, 2H, J=7.0, NCH2), 2.05-1.95 (m, 2H, NCH2CH2), 1.3-40-1.25 (m, 2H, CH2CH3), 0.97 (t, 3H, J=7.4, CH3).

### Example 6.34

### Preparation of 1-(Cyclopropylmethyl)-1H-benzotriazole-5-carboxylic acid

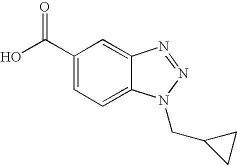

1-(Cyclopropylmethyl)-1H-benzotriazole-5-carboxylic acid was prepared in a similar manner as described in Example 6.32 using cyclopropylmethyl bromide. m/z (ES+): 218 [M+H]+. ¹H NMR (CD₃OD): 8.46 (s, 1H, C(4)-H), 8.03 (dd, 1H, J1=8.9, J2=0.9, C(6)-H), 7.79 (d, 1H, J=8.9, C(7)-H), 4.60 (d, 2H, J=7.6, NCH2), 1.55-1.50 (m, 1H, NCH2CH), 0.70-0.60 (m, 2H), 0.60-0.50 (m, 2H).

### Example 6.35

### Preparation of 1-(But-2-ynyl)-1H-benzotriazole-5-carboxylic acid

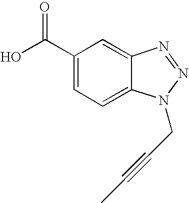

1-(But-2-ynyl)-1H-benzotriazole-5-carboxylic acid was prepared in a similar manner as described in Example 6.32 using 1-bromo-but-2-yne. m/z (ES+): 216 [M+H]+. ¹H NMR (CD₃OD): 8.79 (s, 1H, C(4)-H), 8.21 (d, 1H, J=9.4, C(6)-H), 7.91 (d, 1H, J=8.8, C(7)-H), 4.97 (s, 2H, NCH2), 1.90 (s, 3H, CH3).

### Example 6.36

### Preparation of 1-(4′-Methyl-pentyl)-1H-benzotriazole-5-carboxylic acid

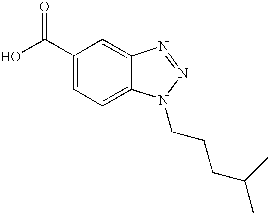

1-(4′-Methyl-pentyl)-1H-benzotriazole-5-carboxylic acid was prepared in a similar manner as described in Example 6.32 using 1-bromo-4-methyl-pentane. m/z (ES+): 248 [M+H]+. ¹H NMR (CD₃OD): 8.69 (dd, 1H, J1=1.4, J2=0.7, C(4)-H), 8.21 (dd, 1H, J1=8.8, J2=1.4, C(6)-H), 7.86 (dd, 1H, J1=8.8, J2=0.7, C(7)-H), 4.75 (t, 2H, J=7.0, NCH2), 2.10-2.00 (m, 2H, NCH2CH2), 1.65-1.55 (m, 1H, CH(CH3)2), 1.30-1.15 (m, 2H, CH2CH), 0.89 (d, 6H, J=6.8, CH3).

### Example 6.37

### Preparation of 1-(3′-Methyl-butyl)-1H-benzotriazole-5-carboxylic acid

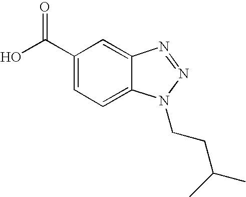

1-(3′-Methyl-butyl)-1H-benzotriazole-5-carboxylic acid was prepared in a similar manner as described in Example 6.32 using 1-bromo-3-methyl-butane. ¹H NMR (CD₃OD): 8.69 (d, 1H, 3=0.8, C(4)-H), 8.21 (dd, 1H, J1=8.8, J2=1.4, C(6)-H), 7.86 (dd, 1H, J1=8.8, J2=0.7, C(7)-H), 4.79 (t 2H, J=7.4, NCH2), 1.92 (q like, 2H, J=7.2, NCH2CH2), 1.60-1.50 (m, 1H, CH(CH3)2), 1.00 (d, 6H, J=6.4, CH3).

### Example 7

In Vivo Animal Model

The utility of the compound of the present invention as a medical agent in the prophylaxis and treatment of a high total cholesterol/HDL-cholesterol ratio and conditions relating thereto is demonstrated by the activity of the compound in lowering the ratio of total cholesterol to HDL-cholesterol, in elevating HDL-cholesterol, or in protection from atherosclerosis in an in viva pig model. Pigs are used as an animal model because they reflect human physiology, especially lipid metabolism, more closely than most other animal models. An illustrative in vivo pig model not intended to be limiting is presented here.

Yorkshire albino pigs (body weight 25.5±4 kg) are fed a saturated fatty acid rich and cholesterol rich (SFA-CHO) diet during 50 days (1 kg chow 35 kg⁻¹ pig weight), composed of standard chow supplemented with 2% cholesterol and 20% beef tallow [Royo T et al., European Journal of Clinical Investigation (2000) 30:843-52; which disclosure is hereby incorporated by reference in its entirety]. Saturated to unsaturated fatty acid ratio is modified from 0.6 in normal pig chow to 1.12 in the SFA-CHO diet. Animals are divided into two groups, one group (n=8) fed with the SFA-CHO diet and treated with placebo and one group (n=8) fed with the SFA-CHO diet and treated with the compound (3.0 mg kg⁻¹). Control animals are fed a standard chow for a period of 50 days. Blood samples are collected at baseline (2 days after the reception of the animals), and 50 days after the initiation of the diet. Blood lipids are analyzed. The animals are sacrificed and necropsied.

Alternatively, the foregoing analysis comprises a plurality of groups each treated with a different dose of the compound. Preferred said doses are selected from the group consisting of: 0.1 mg kg⁻¹, 0.3 mg kg⁻¹, 1.0 mg kg⁻¹, 3.0 mg kg⁻¹, 10 mg kg⁻¹, 30 mg kg⁻¹ and 100 mg kg⁻¹. Alternatively, the foregoing analysis is carried out at a plurality of timepoints. Preferred said timepoints are selected from the group consisting of 10 weeks, 20 weeks, 30 weeks, 40 weeks, and 50 weeks.

HDL-Cholesterol

Blood is collected in trisodium citrate (3.8%, 1:10). Plasma is obtained after centrifugation (1200 g 15 min) and immediately processed. Total cholesterol, HDL-cholesterol, and LDL-cholesterol are measured using the automatic analyzer Kodak Ektachem DT System (Eastman Kodak Company, Rochester, N.Y., USA). Samples with value parameters above the range are diluted with the solution supplied by the manufacturer and then re-analyzed. The total cholesterol/HDL-cholesterol ratio is determined. Comparison is made of the level of HDL-cholesterol between groups. Comparison is made of the total cholesterol/HDL-cholesterol ratio between groups.

Elevation of HDL-cholesterol or reduction of the total cholesterol/HDL-cholesterol ratio on administration of the compound is taken as indicative of the compound having the aforesaid utility.

Atherosclerosis

The thoracic and abdominal aortas are removed intact, opened longitudinally along the ventral surface, and fixed in neutral-buffered formalin after excision of samples from standard sites in the thoracic and abdominal aorta for histological examination and lipid composition and synthesis studies. After fixation, the whole aortas are stained with Sudan IV and pinned out flat, and digital images are obtained with a TV camera connected to a computerized image analysis system (Image Pro Plus; Media Cybernetics, Silver Spring, Md.) to determine the percentage of aortic surface involved with atherosclerotic lesions [Gerrity R G et al, Diabetes (2001) 50:1654-65; Cornhill J F et al, Arteriosclerosis, Thrombosis, and Vascular Biology (1985) 5:415-26; which disclosures are hereby incorporated by reference in their entirety]. Comparison is made between groups of the percentage of aortic surface involved with atherosclerotic lesions.

Reduction of the percentage of aortic surface involved with atherosclerotic lesions on administration of the compound is taken as indicative of the compound having the aforesaid utility.

### Example 8

Receptor Binding Assay

In addition to the methods described herein, another means for evaluating a test compound is by determining binding affinities to the RUP38 receptor. This type of assay generally requires a radiolabelled ligand to the RUP38 receptor. Absent the use of known ligands for the RUP38 receptor and radiolabels thereof, compounds of Formula (I) can be labelled with a radioisotope and used in an assay for evaluating the affinity of a test compound to the RUP38 receptor.

A radiolabelled RUP38 compound of Formula (I) can be used in a screening assay to identify/evaluate compounds. In general terms, a newly synthesized or identified compound (i.e., test compound) can be evaluated for its ability to reduce binding of the “radiolabelled compound of Formula (I)” to the RUP38 receptor. Accordingly, the ability to compete with the “radio-labelled compound of Formula (a)” or Radiolabelled RUP38 Ligand for the binding to the RUP38 receptor directly correlates to its binding affinity of the test compound to the RUP38 receptor.

Assay Protocol for Determining Receptor Binding for RUP38:

A. RUP38 Receptor Preparation

293 cells (human kidney, ATCC), transiently transfected with 10 ug human RUP38 receptor and 60 ul Lipofectamine (per 15-cm dish), are grown in the dish for 24 hours (75% confluency) with a media change and removed with 10 ml/dish of Hepes-EDTA buffer (20 mM Hepes+10 mM EDTA, pH 7.4). The cells are centrifuged in a Beckman Coulter centrifuge for 20 minutes, 17,000 rpm (JA-25.50 rotor). Subsequently; the pellet is resuspended in 20 mM Hepes+1 mM EDTA, pH 7.4 and homogenized with a 50-ml Dounce homogenizer and again centrifuged. After removing the supernatant, the pellets are stored at −80° C., until used in binding assay. When used in the assay, membranes are thawed on ice for 20 minutes and then 10 mL of incubation buffer (20 mM Hepes, 1 mM MgCl₂, 100 mM NaCl, pH 7.4) added. The membranes are vortexed to resuspend the crude membrane pellet and homogenized with a Brinkmann PT-3100 Polytron homogenizer for 15 seconds at setting 6. The concentration of membrane protein is determined using the BRL Bradford protein assay.

B. Biding Assay

For total binding, a total volume of 50 ul of appropriately diluted membranes (diluted in assay buffer containing 50 mM Tris HCl (pH 7.4), 10 mM MgCl₂, and 1 mM EDTA; 5-50 ug protein) is added to 96-well polyproylene microtiter plates followed by addition of 100 ul of assay buffer and 50 ul of Radiolabelled RUP38 Ligand. For nonspecific binding, 50 ul of assay buffer is added instead of 100 ul and an additional 50 ul of 10 uM cold RUP38 is added before 50 ul of Radiolabelled RUP38 Ligand is added. Plates are then incubated at room temperature for 60-120 minutes. The binding reaction is terminated by filtering assay plates through a Microplate Devices GF/C Unifilter filtration plate with a Brandell 96-well plate harvestor followed by washing with cold 50 mM Tris HCl, pH 7.4 containing 0.9% NaCl. Then, the bottom of the filtration plate are sealed, 50 ul of Optiphase Supermix is added to each well, the top of the plates are sealed, and plates are counted in a Trilux MicroBeta scintillation counter. For compound competition studies, instead of adding 100 ul of assay buffer, 100 ul of appropriately diluted test compound is added to appropriate wells followed by addition of 50 ul of Radiolabelled RUP38 Ligand.

C. Calculations

The test compounds are initially assayed at 1 and 0.1 μM and then at a range of concentrations chosen such that the middle dose would cause about 50% inhibition of a Radio-RUP38 Ligand binding (i.e., IC₅₀). Specific binding in the absence of test compound (B_{O}) is the difference of total binding (B_{T}) minus non-specific binding (NSB) and similarly specific binding (in the presence of test compound) (B) is the difference of displacement binding (B_{D}) minus non-specific binding (NSB). IC₅₀ is determined from an inhibition response curve, logit-log plot of % B/B_{O} vs concentration of test compound.

Kᵢ is calculated by the Cheng and Prustoff transformation:
Kᵢ=IC₅₀/(1+[L]/K_{D})

where [L] is the concentration of a Radio-RUP38 Ligand used in the assay and K_{D} is the dissociation constant of a Radio-RUP38 Ligand determined independently under the same binding conditions.

Throughout this application, various publications, patents and published patent applications are cited. The disclosures of these publications, patents and published patent applications referenced in this application are hereby incorporated by reference in their entirety into the present disclosure. Modifications and extension of the disclosed inventions that are within the purview of the skilled artisan are encompassed within the above disclosure and the claims that follow.

Although a variety of expression vectors are available to those in the art, for purposes of utilization for both the endogenous and non-endogenous human GPCRs, it is most preferred that the vector utilized be pCMV. This vector was deposited with the American Type Culture Collection (ATCC) on Oct. 13, 1998 (10801 University Blvd., Manassas, Va. 20110-2209 USA) under the provisions of the Budapest Treaty for the International Recognition of the Deposit of Microorganisms for the Purpose of Patent Procedure. The DNA was tested by the ATCC and determined to be viable. The ATCC has assigned the following deposit number to pCMV: ATCC #203351.

1. A compound of Formula (I): 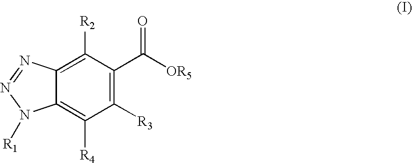
wherein:
R₁ is C₁₋₈ alkyl, C₃₋₆ cycloalkyl or C₁₋₆ haloalkyl, wherein the C₁₋₈ alkyl, C₃₋₆ cycloalkyl and C₁₋₆ haloalkyl groups are optionally substituted with 1, 2, 3 or 4 substituents selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, aryl, substituted aryl, C₁₋₆ dialkylamino, carbo C₁₋₆ alkoxy, carboxy, cyano, C₃₋₄ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, heteroaryl, heterocyclyl, hydroxyl, nitro and thiol;
R₂, R₃ and R₄ are each independently selected from the group consisting of H, C₁₋₆acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro and thiol; and
R₅ is H or C₁₋₆ alkyl; or
a pharmaceutically acceptable salt solvate or hydrate thereof;
provided that:
a) when R₅ is ethyl, and R₂, R₃ and R₄ are H then R₁ is not methyl or triphenylmethyl;
b) when R₅ is n-pentyl, and R₂, R₃ and R₄ are H then R₁ is not n-butyl;
c) when R₅ is methyl, and R₂, R₃ and R₄ are H then R₁ is not pyrrolidin-1-ylmethyl, 3-tert-butyl-2-hydroxy-5-methyl-benzyl, methyl, or dimethylaminomethyl;
d) when R₅ is methyl, R₂ is carbomethoxy and R₃ and R₄ are both H then R₁ is not methyl;
e) when R₂, R₃, R₄ and R₅ are all H then R₁ is not 2-amino-2-carboxy-ethyl, pyrrolidin-1-ylmethyl, isopropyl, methyl, benzyl, n-butyl, or carboxymethyl; and
f) when R₂, R₄, and R₅ are all H and R₃ is methoxy then R₁ is not methyl.

2. A compound according to claim 1 wherein:
R₁ is C₃₋₆ cycloalkyl or C₁₋₆ haloalkyl, wherein each C₃₋₆ cycloalkyl and C₁₋₆ haloalkyl group is optionally substituted with 1, 2, 3, or 4 substituents selected form the group consisting of C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro and thiol;
R₂, R₃ and R₄ are each independently selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₄ haloalkoxy, C₁₋₆ haloalkyl, C₁₋₆ haloalkylsulfinyl, C₁₋₆ haloalkylsulfonyl, C₁₋₆ haloalkylthio, hydroxyl, nitro and thiol; and
R₅ is H or C₁₋₆ alkyl; or
a pharmaceutically acceptable salt, solvate or hydrate thereof.

3. The compound according to claim 1 wherein R₅ is C₁₋₆ alkyl.

4. The compound according to claim 1 wherein R₅ is H.

5. The compound according to claim 1 wherein R₂, R₃ and R₄ are each independently H or halogen.

6. The compound according to claim 1 wherein R₂, R₃ and R₄ are each independently H or F.

7. The compound according to claim 1 wherein R₁ is C₁₋₈ alkyl optionally substituted with substituents selected from the group consisting of C₂₋₆ alkenyl, C₁₋₆ alkoxy, C₂₋₆ alkynyl, C₁₋₆ alkylsulfinyl, C₁₋₆ alkylsulfonyl, C₁₋₆ alkylthio, aryl, substituted aryl, C₃₋₆ cycloalkyl, halogen, C₁₋₆ haloalkoxy, C₁₋₆ haloalkylsulfinyl, C₁₋₆ haloalkylsulfonyl, C₁₋₆ haloalkylthio, heteroaryl, heterocyclyl, and hydroxyl.

8. The compound according to claim 1 wherein R₁ is selected from the group consisting of 2-butyl, 3-pentyl, 1-propyl, t-butyl, 1-butyl, 4-Methyl-pentyl, 3-methyl-butyl, 1,3-dimethyl-butyl, 3,3-dimethyl-butyl, 1-heptyl, ethyl, 2,2-dimethyl-propyl, and 1-pentyl.

9. The compound according to claim 1 wherein R₁ is selected from the group consisting of 3-methoxy-benzyl, 4-methoxy-benzyl, 4-methoxy-phenyl ethyl, 3-methoxy-phenyl ethyl, 3,5-difluorobenzyl, and benzhydryl.

10. The compound according to claim 1 wherein R₁ is selected from the group consisting of 3-isopropoxypropyl, tetrahydro-furan-2-ylmethyl, 2-methoxy-ethyl, 2-ethylsulfanyl-ethyl, 3-hydroxy-propyl, allyl, cyclopropylmethyl, but-2-ynyl, 2-methoxy-1-methyl-ethyl, 2-hydroxy-1-hydroxymethyl-ethyl, 2-ethoxy-ethyl, and 1,2-dimethyl-propyl.

11. The compound according to claim 1 wherein R₁ is selected from the group consisting of cyclopentyl, cyclohexyl, cyclopropyl, and cyclobutyl.

12. The compound according to claim 1 selected from the group consisting of:
1-Cyclopentyl-1H-benzotriazole-5-carboxylic acid;
1-(2′-Butyl)-1H-benzotriazole-5-carboxylic acid;
1-(3′-Pentyl)-1H-benzotriazole-5-carboxylic acid;
1-Cyclohexyl-1H-benzotriazole-5-carboxylic acid
1-Propyl-1H-benzotriazole-5-carboxylic acid;
1-Cyclopropyl-1H-benzotriazole-5-carboxylic acid;
1-(3′-Isopropoxy-propyl)-1H-benzotriazole-5-carboxylic acid;
1-(Tetrahydro-furan-2′-ylmethyl)-1H-benzotriazole-5-carboxylic acid;
1-Cyclobutyl-1H-benzotriazole-5-carboxylic acid;
1-(2-Methoxy-ethyl)-1H-benzotriazole-5-carboxylic acid;
1-(3′Methoxybenzyl)-1H-benzotriazole-5-carboxylic acid;
1-(4′Methoxybenzyl)-1H-benzotriazole-5-carboxylic acid;
1-[2′-(4″-Methoxy-phenyl)-ethyl]-1H-benzotriazole-5-carboxylic acid;
1-[2′-(3″-Methoxy-phenyl)-ethyl]-1H-benzotriazole-5-carboxylic acid;
1-(3′,5′-Difluorobenzyl)-1H-benzotriazole-5-carboxylic acid;
1-(2-Ethylsulfanyl-ethyl)-1H-benzotriazole-5-carboxylic acid;
1-t-Butyl-1H-benzotriazole-5-carboxylic acid;
1-(3′-Hydroxy-propyl)-1H-benzotriazole-5-carboxylic acid;
1-(1′,3′-Dimethyl-butyl)-1H-benzotriazole-5-carboxylic acid;
1-(3′,3′-Dimethyl-butyl)-1H-benzotriazole-5-carboxylic acid;
1-Heptyl-1H-benzotriazole-5-carboxylic acid;
1-(2′-Methoxy-1′-methyl-ethyl)-1H-benzotriazole-5-carboxylic acid;
1-(2′-Hydroxy-1′-hydroxymethyl-ethyl)-1H-benzotriazole-5-carboxylic acid;
1-Ethyl-1H-benzotriazole-5-carboxylic acid;
1-Pentyl-1H-benzotriazole-5-carboxylic acid;
1-(2′,2′-Dimethyl-propyl)-1H-benzotriazole-5-carboxylic acid;
1-(2′-Ethoxy-ethyl)-1H-benzotriazole-5-carboxylic acid;
1-(1′,2′-Dimethyl-propyl)-1H-benzotriazole-5-carboxylic acid;
1-Benzhydryl-1H-benzotriazole-5-carboxylic acid;
1-Allyl-1H-benzotriazole-5-carboxylic acid;
1-Butyl-1H-benzotriazole-5-carboxylic acid;
1-(Cyclopropylmethyl)-1H-benzotriazole-5-carboxylic acid;
1-(But-2-ynyl)-1H-benzotriazole-5-carboxylic acid;
1-(4′-Methyl-pentyl)-1H-benzotriazole-5-carboxylic acid; and
1-(3′-Methyl-butyl)-1H-benzotriazole-5-carboxylic acid; or
a pharmaceutically acceptable salt, solvate or hydrate thereof.

13. A pharmaceutical composition comprising a compound according to
Formula (I): 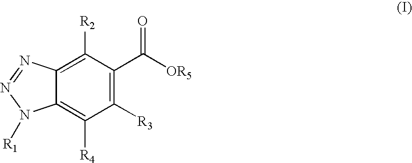
wherein:
R₁ is H, C₁₋₈ alkyl, C₃₋₆ cycloalkyl or C₁₋₆ haloalkyl, wherein each C₁₋₆ alkyl, C₃₋₆ cycloalkyl and C₁₋₆ haloalkyl group is optionally substituted with 1, 2, 3, or 4 substituents selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₄ cycloalkyl, C₁₋₆ dialkylcarboxamido, halogen, C₁₋₄ haloalkoxy, C₁₋₄ haloalkyl, C₁₋₆ haloalkylsulfinyl, C₁₋₆ haloalkylsulfonyl, C₁₋₆ haloalkylthio, hydroxyl, nitro and thiol;
R₂, R₃ and R₄ are each independently selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamido, C₂₋₆ alkynyl, C₁₋₆ alkylsulfinyl, C₁₋₆ alkylsulfonyl, C₁₋₆ alkylthio, C₁₋₆ alkylureyl, amino, C₁₋₆ alkylamino, C₁₋₆ dialkylamino, carbo C₁₋₆ alkoxy, carboxy, cyano, C₃₋₆ cycloalkyl, C₁₋₆ dialkylcarboxamido, halogen, C₁₋₄ haloalkoxy, C₁₋₄ haloalkyl, C₁₋₄ haloalkylsulfinyl, C₁₋₄ haloalkylsulfonyl, C₁₋₆ haloalkylthio, hydroxyl, nitro and thiol; and
R₅ is H or C₁₋₆ alkyl; or
a pharmaceutically acceptable salt, solvate or hydrate thereof, in combination with a pharmaceutically acceptable carrier.

14. A pharmaceutical composition according to claim 13 further comprising an agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

15. (canceled)

16. (canceled)

17. (canceled)

18. (canceled)

19. (canceled)

20. A method of treatment of a metabolic-related disorder comprising administering to an individual in need of such treatment a therapeutically effective amount of a pharmaceutical composition according to claim 13.

21. A method according to claim 20 wherein said metabolic-related disorder is selected from the group consisting of dyslipidemia, atherosclerosis, coronary heart disease, insulin resistance and type 2 diabetes.

22. (canceled)

23. (canceled)

24. (canceled)

25. (canceled)

26. A method of treatment of a metabolic-related disorder comprising administering to an individual in need of such treatment a therapeutically effective amount of a compound according to claim 1.

27. A method according to claim 26 wherein said metabolic-related disorder is selected from the group consisting of dyslipidemia, atherosclerosis, coronary heart disease, insulin resistance and type 2 diabetes.

28. The pharmaceutical composition according to claim 13 wherein said compound is selected from the group consisting of:
1-Isopropyl-1H-benzotriazole-5-carboxylic acid;
1-Cyclopentyl-1H-benzotriazole-5-carboxylic acid;
1-(2′-Butyl)-1H-benzotriazole-5-carboxylic acid;
1-(3′-Pentyl)-1H-benzotriazole-5-carboxylic acid;
1-Cyclohexyl-1H-benzotriazole-5-carboxylic acid;
1-Benzyl-1H-benzotriazole-5-carboxylic acid;
1-Propyl-1H-benzotriazole-5-carboxylic acid;
1-Cyclopropyl-1H-benzotriazole-5-carboxylic acid;
1-(3′-Isopropoxy-propyl)-1H-benzotriazole-5-carboxylic acid;
1-(Tetrahydro-furan-2′-ylmethyl)-1H-benzotriazole-5-carboxylic acid;
1-Cyclobutyl-1H-benzotriazole-5-carboxylic acid;
1-(2-Methoxy-ethyl)-1H-benzotriazole-5-carboxylic acid;
1-(3′Methoxybenzyl)-1H-benzotriazole-5-carboxylic acid;
1-(4′Methoxybenzyl)-1H-benzotriazole-5-carboxylic acid;
1-[2′-(4″-Methoxy-phenyl)-ethyl]-1H-benzotriazole-5-carboxylic acid;
1-[2′-(3″-Methoxy-phenyl)-ethyl]-1H-benzotriazole-5-carboxylic acid;
1-(3′,5′-Difluorobenzyl)-1H-benzotriazole-5-carboxylic acid;
1-(2′-Ethylsulfanyl-ethyl)-1H-benzotriazole-5-carboxylic acid;
1-t-Butyl-1H-benzotriazole-5-carboxylic acid;
1-(3′-Hydroxy-propyl)-1H-benzotriazole-5-carboxylic acid;
1-(1′,3′-Dimethyl-butyl)-1H-benzotriazole-5-carboxylic acid;
1-(3′,3′-Dimethyl-butyl)-1H-benzotriazole-5-carboxylic acid;
1-Heptyl-1H-benzotriazole-5-carboxylic acid;
1-(2′-Methoxy-1′-methyl-ethyl)-1H-benzotriazole-5-carboxylic acid;
1-(2′-Hydroxy-1′-hydroxymethyl-ethyl)-1H-benzotriazole-5-carboxylic acid;
1-Ethyl-1H-benzotriazole-5-carboxylic acid;
1-Pentyl-1H-benzotriazole-5-carboxylic acid;
1-(2′,2′-Dimethyl-propyl)-1H-benzotriazole-5-carboxylic acid;
1-(2′-Ethoxy-ethyl)-1H-benzotriazole-5-carboxylic acid;
1-1′,2′-Dimethyl-propyl)-1H-benzotriazole-5-carboxylic acid;
1-Benzhydryl-1H-benzotriazole-5-carboxylic acid;
1-Allyl-1H-benzotriazole-5-carboxylic acid;
1-Butyl-1H-benzotriazole-5-carboxylic acid;
1-(Cyclopropylmethyl)-1H-benzotriazole-5-carboxylic acid;
1-(But-2-ynyl)-1H-benzotriazole-5-carboxylic acid;
1-(4′-Methyl-pentyl)-1H-benzotriazole-5-carboxylic acid; and
1-(3′-Methyl-butyl)-1H-benzotriazole-5-carboxylic acid; or
a pharmaceutically acceptable salt, solvate or hydrate thereof.


SEQUENCE LISTING (4 sequences)

SEQ ID NO: 1 - DNA, 1164 bp, Homo sapien
atgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaactttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaa

SEQ ID NO: 2 - protein, 387 aa, Homo sapien
MNRHHLQDHFLEIDKKNCCVFRDDFIAKVLPPVLGLEFIFGLLGNGLALWIFCFHLKSWKSSRIFLFNLAVADFLLIICLPFVMDYYVRRSDWNFGDIPCRLVLFMFAMNRQGSIIFLTVVAVDRYFRVVHPHHALNKISNWTAAIISCLLWGITVGLTVHLLKKKLLIQNGPANVCISFSICHTFRWHEAMFLLEFLLPLGIILFCSARIIWSLRQRQMDRHAKIKRAITFIMVVAIVFVICFLPSVVVRIRIFWLLHTSGTQNCEVYRSVDLAFFITLSFTYMNSMLDPVVYYFSSPSFPNFFSTLINRCLQRKMTGEPDNNRSTSVELTGDPNKTRGAPEALMANSGEPWSPSYLGPTSNNHSKKGHCHQEPASLEKQLGCCIE

SEQ ID NO: 3 - DNA, 1092 bp, Homo sapien
atgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctccttaa

SEQ ID NO: 4 - protein, 363 aa, Homo sapien [sequence omitted]